(19) United States
(12) Patent Application Publication
(10) Pub. No.: US 2004/0229290 A1
(43) Pub. Date: Nov. 18, 2004

(54) Protein design for receptor-ligand recognition and binding

(75) Inventors: Homme W. Hellinga, Durham, NC (US); Loren L. Looger, Madison, AL (US); Mary A. Dwyer, Chapel Hill, NC (US)

Correspondence Address: NIXON & VANDERHYE, PC, 1100 N GLEBE ROAD, 8TH FLOOR, ARLINGTON, VA, 22201-4714, US

(73) Assignee: DUKE UNIVERSITY, Durham, NC

(21) Appl. No.: 10/840,796

(22) Filed: May 7, 2004

Related U.S. Application Data
(60) Provisional application No. 60/468,270, filed on May 7, 2003.

(51) Int. Cl. G01N 33/53; G06F 19/00; G01N 33/48; G01N 33/50

(52) U.S. Cl. 435/7.1; 702/19

(57) ABSTRACT

We describe processes for the protein structure-based design or redesign of receptor-ligand interfaces (ligand-binding sites) in which a ligand is recognized and bound. Receptors designed in this manner can then be synthesized artificially or naturally, or used to engineer cells, tissues, or organisms. They can be further evaluated by empirical methods (e.g., ligand recognition and binding, signaling, catalysis), subjected to further improvement, and/or the process can be iterated in multiple cycles (e.g., consideration of quantitative structure-activity relationship data).

### CROSS-REFERENCE TO RELATED APPLICATION

This application claims the benefit of provisional U.S. Appln. No. 60/468,270, filed May 7, 2003; which is incorporated by reference herein.

### FEDERAL GOVERNMENT SUPPORT

This invention was made with federal government support under grant GM049871 awarded by the National Institutes of Health, grant N0014-01-1-0238 awarded by the Office of Naval Research, and grant F49620-02-0063 awarded by the Defense Advanced Research Project Agency. The U.S. Government has certain rights in the invention.

### FIELD OF THE INVENTION

Formation of a complex between a receptor and its ligand is fundamental to biological processes at the molecular level. Manipulation of molecular recognition between a ligand and its receptor is therefore important for study of biological phenomena (1) and has numerous applications, including, but not limited to, construction of improved or novel enzymes (2-5), biosensors (6, 7), genetic circuits (8), signal transduction pathways (9), and chiral separations (10). Preliminary results were published by us in Looger et al. (11).

### BACKGROUND OF THE INVENTION

The most commonly used methods for altering specificities are empirical, using either the immune system to generate antibodies (13), directed evolution or gene shuffling (14), or screening of large libraries for altered functionality (15). These approaches lose in generality either because they are limited to a particular class of proteins (antibodies), or because of constraints in the sequence diversity and methodologies available (selection by directed evolution or gene shuffling, library screening). In practice, it is typically possible to screen protein libraries fully degenerate at no more than 10, or certainly 15, positions (16). Structure-based, rational design techniques potentially offer enormous generality for manipulating protein structure and function (17). Generality arises from (i) the ability to describe any chemical structure (scaffold or target ligand), and (ii) the use of computational algorithms that can address combinatorial search spaces that vastly exceed those addressable empirically (16).

The general principles for the formation of specific complexes are understood in considerable depth (18), and involve a lock-and-key fit between ligand and receptor, the structure of which is determined primarily by short-range interactions (e.g., steric contacts, hydrogen bonds). Complex formation is thermodynamically driven primarily by hydrophobic effects (19), long-range electrostatics (20, 21), and possibly by differences between protein interiors and solvent in the strength of the short-range interactions (22). Difficulties in structure-based computational design arise from limitations in the description of the molecular interactions (23) and the combinatorial complexity of the problem (16, 24). Despite notable advances in the rational manipulation of protein sequence and stability using automated computational design tools (16), prior to this invention the computational design of ligand-binding properties has been limited to metal centers (5), changes in binding specificity in which much of the chemical character of the wild-type ligand is retained (2, 9, 25) or larger changes in binding specificity which resulted in relatively weak binding (3).

In comparison to the selection of enzymes by catalytic antibodies, this invention has several other advantages. The ligand, which is a transition-state analog for the target chemical reaction, must possess sufficient stability and antigenicity to induce an antibody response. But it must be nontoxic to the immunized animal and not cause undesirable biological effects. In contrast, the design algorithms of this invention does not require chemical synthesis of the ligand or its administration to an animal because the ligand can be manipulated in silico. The efficiency of this invention is shown by the proportion of designs that successfully bind ligand and/or catalyze a reaction, whereas a large number of hybridomas are typically screened to select an antibody with modest catalytic activity. Furthermore, the proteins designed by this invention can be synthesized with one or more non-natural residues which form peptide bonds, side chains thereof, post-translational modifications, and combinations thereof instead of relying on anti-body-producing cells which are capable of only natural protein synthesis.

### SUMMARY OF THE INVENTION

It is an objective of the invention to provide processes for the protein structure-based design or redesign of receptor-ligand interfaces (ligand-binding sites) in which a ligand is recognized and bound. Receptors designed in this manner can then be manufactured or used to engineer cells, tissues, or organisms. They can be further evaluated by empirical methods (e.g., ligand recognition and binding, gene expression, signaling pathways, catalysis), subjected to further improvement, and/or the process can be iterated in multiple cycles (further comprising a consideration of quantitative structure-activity relationship data).

The invention thus relates to a process for protein design in accordance with spatial and energy relationships between a proteinaceous receptor and a ligand. The process can comprise (a) generating a collection of ligand poses to provide a Docking Zone that represents potential conformations and degrees of freedom of the ligand relative to the receptor, (b) generating a collection of amino acid side-chain conformations on the backbone of the receptor to provide an Evolving Zone; (c) calculating a cost function (e.g., atomic interaction(s) between the ligand poses of the Docking Zone and the amino acid side chains of the Evolving Zone, and between amino acid side chains of the Evolving Zone); (d) generating a collection of candidate receptor designs with ligand binding sites by selecting from combinations of the ligand poses and the amino acid side chains one or more of the combinations that corresponds to optimal or near-optimal values of the cost function; and optionally (e) rank-ordering candidate receptor designs of the collection resulting from (d) by a fitness metric to identify one or more candidate receptor designs that potentially binds to the ligand. Binding to the ligand of the one or more candidate receptor designs can then be confirmed; alternatively, the ligand may be an analog which is bound or a reactive substrate or product of an enzyme.

Some improvements of the invention over the prior art are using the Docking Zone and the Evolving Zone in calculating atomic interactions between receptor and ligand (i.e., potential function), selecting one or more pairs of receptor-ligand from a subset of all possible combinations, evaluating the hydrogen bond inventory of the ligand and/or binding surface inventory of the receptor-ligand interaction, and algorithms to rank-order and select pairs of ligands and mutated receptors. Further mutations in the receptor may be introduced outside its ligand binding site to stabilize the protein, to increase affinity for ligand, to improve catalysis, or a combination thereof because the further mutations act on residues in the Evolving Zone.

The process can be implemented as a computer system or stored on tangible medium. Protein designed by the invention and made by chemical synthesis or translation; nucleic acid encoding that protein; an expression vector comprised of that nucleic acid; and an engineered cell, tissue, or non-human organism are other embodiments of the invention.

Further aspects of the invention will be apparent to a person skilled in the art from the following description and claims, and generalizations thereto.

### DESCRIPTION OF THE DRAWINGS

FIG. 1 shows an embodiment of the invention. The flowchart highlights major stages in the Receptor Design algorithm: (i) preparation of target ligand, including force field and structural descriptions; (ii) preparation of design scaffold, including identification of target binding site, docking grid, and docking hull; (iii) construction of CLIPs (Compatible LIgand Poses), to represent the ensemble of all possible compatible poses of the target ligand within the target binding site; (iv) generation of a family of complementary surfaces against the CLIPs; and (v) refinement of this family of complementary surfaces by well search of related sequences, ranking by receptor-ligand interface estimators, and design cycle feedback from experimental characterization of designed receptors.

FIG. 2 shows the conformational equilibrium of the periplasmic binding protein (PBP) superfamily, and target ligands and structurally-related compounds. (A) Ribose-binding protein is shown as representative of the protein superfamily. Ribose binding mediates a transition from an open (left) to a closed (right) conformation (62, 86). The protein has two domains (I, amino terminal; II, carboxy terminal) linked by a hinge region (H). Fluorescence intensity changes of an environmentally sensitive, thiol-reactive fluorescent dye (shown as a solid sphere near the hinge region) coupled to a mutant cysteine at position 265 monitor ligand binding (7). Calculations use the closed structure, mutating the PCS residues, and docking the target ligands into the convex hull (shown only as edges). (B) Structures of target ligands and structurally related decoys used to probe the specificity of the designed receptors.

FIG. 3 shows stereo views of representative designed ligand-binding sites: (A) TNT.R3; (B) Lac.R1; (C) Lac.H1; (D) Stn.A1 (dashed line: hydrogen bonds between protein and ligand; numbers: side chains close to the ligand). TNT.R3 and Lac.R1 are presented in the same orientation, illustrating the adaptability of the RBP scaffold to bind different ligands. The Lac.R1 and Lac.H1 structures illustrate that the same ligand can be bound by sites designed in different scaffolds.

FIG. 4 shows fluorescence data for a representative designed receptor Lac.R1. (A) Fluorescence emission spectra for apo (closed circle) and L-lactate-saturated (open circle) protein solution. (B) Fluorescence emission intensity at 470 nm is shown as a function of L-lactate concentration. The fluorescence titration profile is fit to a single-site binding isotherm (7).

FIG. 5 shows thermostability data for a representative subset of designed receptors. Experiments were conducted in 20 mM sodium phosphate and 150 mM sodium chloride, pH 7.0; protein concentration was 10 mM. Ellipticity was monitored at 222 nm. Measured Tₘs for mutants: TNT.A1 (circle), 52° C.; TNT.R1, 42° C.; TNT.R2 (square), 54° C.; TNT.H1, 46° C.; Lac. A3 (diamond), 46° C.; Lac.G2, 50° C.; Lac.H1 (triangle), 45° C. These results show that the mid-point transitions fall within 2-15° C. of the wild-type proteins (wild-type Tₘs are: RBP, 58° C.; GBP, 59° C.; HBP, 58° C.; ABP, 54° C.; QBP, 62° C.), and that the degree of cooperativity of the designed receptors are similar to the wild-type receptors.

FIG. 6 shows ligand-binding specificity data for the designed receptors: (A) TNT, (B) L-lactate, (C) serotonin, and (D) D-lactate. Almost all of the designed receptors show a stronger affinity for their target ligands relative to structurally-related decoys, consistent with correct modeling of receptor-ligand complex. Results are reported as the free energy difference, ΔΔGb, relative to the target ligand (ΔΔGb=RT In (Kd (decoy)/Kd (target)); ΔΔGb>0 indicates preference for target ligand). RT˜0.6 kcal/mol. A ten-fold difference in affinity corresponds to approximately 1.4 kcal/mol of binding specificity. Target ligands and protein scaffolds are denoted using single-letter abbreviations. Ligands: TNT, T; L-lactate, L; serotonin, S; D-lactate, D. Scaffolds: RBP, R; ABP, A; HBP, H; GBP, G; QBP, Q.

FIG. 7 shows quantitative structure-activity relationships (QSARs) for the ligand-binding affinities of the designed receptors. Calculated affinities are obtained from the model structure of the complex by: log(Kd)=c₁+c₂ΔG_{elec}+c₃A+C₄N_{unsat+c}₅N_{clash}+C₆₁|s-s0|. The linear regression coefficients, c₁ . . . c₆, were obtained by least-squares fit of the experimental data; ΔG_{elec} is an electrostatic contribution (87); A is the nonpolar contact area between receptor and ligand; Nᵤₙₛₐₜ is the number of unsatisfied hydrogen bonds in the ligand; N_{clash} is the number of steric clashes between the ligand and receptor (defined as contacts greater than 5 kcal/mol); s is the ratio of the van der Waals volume of the wild-type ligand to that of the target ligand; s0 is the apparent optimum value of s for a particular ligand, obtained by the least-squares fit. Analogs are modeled to bind in the same mode as the target ligand, constructed by superimposition of the phenyl ring for nitro compounds and the carboxylate moiety for lactate analogs. (A) Independent QSARs for TNT (filled circle, solid line) and L-lactate (open circle, dashed line). For TNT, the least-squares fit parameter vector {s0, c₁, C₂, C₃, C₄, C₅, C₆} is {0.84, −6.2, 0.1, −0.05, 0.5, 2.2, 41.3}; and for L-lactate { 1.76, −6.5, 0.09, −0.04, 0.4, 0, 12.7} (for L-lactate C₅ is undetermined, since there are no steric clashes). (B) Combined QSAR obtained by fitting all ligands simultaneously: TNT (filled circle), TNB (filled square), 2,4-DNT (filled diamond), 2,6-DNT (filled triangle), L-lactate (open circle), D-lactate (open square), pyruvate (open diamond). All nitro compounds and lactate analogs were fit together, with only the parameters s₀ and c₆ being ligand-dependent. The resulting fit is {(0.85, 1.73), −5.2, 0.04, −0.03, 0.02, 0.9, (54, 12)} (s₀ and c₆ are ligand-dependent: the first number refers to the nitro compounds, and the second to the lactate analogs).

FIG. 8 shows a synthetic two-component signal transduction pathway (84). (A) The ligand-bound RBP or GBP (i) interacts with the Trg domain (thick black line) of a chimeric transmembrane histidine kinase, Trz (ii), resulting in autophosphorylation of the EnvZ domain (grey line), and phosphate transfer to OmpR (iii), which then binds to the ompC promoter (iv),upregulating lacZ transcription. (B) Response to TNT (circle: TNT.R1; square: TNT.R2; diamond: TNT.R3). (C) Response to sugar (open circle: ribose and wild-type RBP; open square: glucose and wild-type GBP) and L-lactate (filled circle: Lac.R1; filled square: Lac.G1). β-galactosidase activities are reported as the difference in assay end-point absorbances of ligand-stimulated and unstimulated cultures. Sensitivity of E. coli to high TNT or L-lactate concentrations precluded determination of full dose-response curves. There is no response in the absence of receptors or trz.

FIG. 9 shows the chemical structures of soman and related molecules.

FIG. 10 shows another embodiment of the invention. Numbers in the flowchart (A) and molecular drawings (B-E) correspond to processes described herein: panels 1-2, rotational ligand ensemble; panels 3-4, truncated scaffold with alanine surface and convex hull; panels 5-6, placed ligand ensemble; panels 7-8, example of a complementary surface design.

FIG. 11 shows structures of GBP and RBP (domains I and II) with computational models of representative designs (protons are not shown): (A) GBP design PG10, (B) GBP design PG12, and (C) RBP design PR8. Residues selected for alanine-scanning mutagenesis are italicized.

FIG. 12 shows selection of GBP designs (▪, ligand-mediated fluorescent response with experimentally observed affinities as indicated; ●, not tested; ∘, no fluorescent response; x, no protein expression; ⋄, protein precipitation). Designs were chosen from a final list of candidates using a linear optimization procedure that selected a subset corresponding to the intersection of the top 20% ligand van der Waals energy, 50% ligand H-bond energy, with all H-bonds satisfied and with solvent-accessible surface areas less than 15 Å². The designs are shown ranked by the van der Waals energy (E_{vdw}) of the interaction between ligand and receptor, which is a measure of close packing. Inset: correlation between the experimentally determined PMPA affinities and E_{vdw} for the tested designs.

FIG. 13 shows the fluorescent response of fluorescein-labeled PG12 upon titration with PMPA. Inset: emission spectra of protein in the absence (solid line) or presence of 0.5 mM PMPA (dashed line).

FIG. 14 shows the correlation between experimentally determined fragment coupling energy, ΔG_{c}, and the affinity for PMPA, ΔG_{b,PMPA}.

FIG. 15 shows biochemical pathways related to triose phosphate isomerase. (A) Role of TIM in glycolysis, gluconeogenesis, and methylgloxate metabolism (104, 112) (G6P, glucose-6-phosphate; F1,6P₂, fructose-1,6-bisphospate; PFK, phosphofructokinase; MGS, methylglyoxate synthetase). (B) TIM mechanism. (C) Comparison of yeast TIM (110) (flexible loop; catalytic residues; phosphoglycolate) and RBP (62) (I and II, amino terminal and carboxy terminal domains respectively; H, hinge region; ribose) structures.

FIG. 16 shows the predicted structures of RBP-based designs. (A) DHAP-binding receptor D1 (stereo view) with ligand and designed complementary surface residues. (B) NovoTim1.0 (stereo view) with enediolate, catalytic residues, and complementary surface. (C) NovoTim1.2 with a layer of residues surrounding the active site, mutation of which confers near wild-type stability (enediolate; catalytic residues; substrate-binding residues). Also indicated are the mutations isolated by directed evolution of NovoTim1.2 (view hides ²⁶⁴H) that increase enzyme activity (NovoTim1.2.1: Lys76₁Asn, Lys243₁Ser; NovoTim1.2.2: Lys76₁Ala, Glu255₁Val; NovoTim1.2.3: Asp264_{H}Gln; NovoTim1.2.4: Val55₁Ser).

FIG. 17 shows yet another embodiment of the invention. (A) Integration of the algorithms for placing side chains and ligands with predefined geometries (85) to generate partial sites that specify the location and structures of the catalytically active residues, with the design of stereochemically complementary substrate-binding surfaces to design complete active sites. (B) Geometrical definition used to generate placement of the active site residues. Positioning of the catalytic residues (glutamate, histidine, lysine) is shown relative to the plane of the enediolate. The enediolate conformation is designed to minimize phosphate elimination, and is derived from the structure of a phosphoglycolate complex (110). To define the constraints for histidine, a pseudoatom, ψ, was placed midway (circle) between C₁ and C₂. Geometrical constraints are formulated (85) in terms of allowed intervals for bond lengths (l), angles (ω), and torsions (θ) for each residue relative to the enediolate: glutamate, l(C₁,C₆₇ : 2-5 Å), ω₁(C₆₇, C₁, C₂: 107°±30°), ω₂(C₁,C₂,O_{ε1}: 62.30°±30°), θ₁(O₁,C₂, C₁,C₆₇ : 180°±15°), θ₂(C₂,C₁,C_{δ},O_{ε1}: unconstrained), θ₃(C₁,C_{δ},O_{ε1},O_{ε2}: 0°±30°); histidine: l(N_{ε2}, ψ: 2-4 Å), ω₁(Cγ,N_{ε2},ψ: 127.5°), ω₂(N_{ε2},ψ,C₁: 90°), θ₁(C₆₅ ,C_{δ2}, N_{ε2}, ψ: 180°), θ₂(C_{δ2}, N_{ε2}, ψ,C₁: 0°±30°), θ₃(N_{ε2},ψ,C₁,O₁: 0°±45°): lysine: l(O₂,N_{ζ}; 2-5Å), ω₁(O₂,O₂,N_{ζ}: 90°-180°), ω₂(O₂,N_{ζ},C_{ε}: 90°-180°), θ₁(C_{γ},C₂,O₂,N_{ζ}: 180°±90°), θ₂(C₂, O₂,N_{ζ},C_{ε}: unconstrained), θ₃(O₂,N_{ζ}C_{ε},C_{δ}: unconstrained).

FIG. 18 shows the properties of selected designs. (A) Thernostability (reported as mid-point transition, Tₘ, values) monitored by temperature dependence of ellipticity (119) (wild-type RBP, open diamond, Tₘ=58° C.; NovoTim1.0, square, Tₘ=37° C.; NovoTim1.1, diamond, Tₘ=43° C.; NovoTim1.2, circle, Tₘ=52° C.). Steady-state kinetics (Lineweaver-Burke transformation (120)) of NovoTim1.2 for (B) forward (DHAP to GAP) and (C) reverse (GAP to DHAP) reactions. (D) Alanine mutants of catalytic residues (E15, H90, K132) in NovoTim1.2, presented as energy difference diagrams (18) (effects on rate enhancements (k_{cat} changes), stippled; effects on Michaelis complex (K_{M} changes), hashed). (E) pH dependence of k_{cat} for the forward (^{D}k_{cat}, triangles) and reverse (^{G}k_{cat} squares) reactions of NovoTim1.2 (calculated ^{app}pKₐs: forward (6.5, 9.5); reverse (5.9, 9.3)).

### DESCRIPTION OF SPECIFIC EMBODIMENTS OF THE INVENTION

The terms “receptor” and “protein” are used interchangeably herein because the amino acid residues of the receptor are designed by the invention. It is understood, however, that the protein can include non-proteinaceous domains, some of which can contribute to function. The “ligand” is not so limited in its chemical structure because it can be wholly or partially comprised of amino acid, carbohydrate, fatty acid, and small organic or inorganic moieties. Similarly, the terms “binding” and “recognition” are used equivalently. The receptor-ligand nomenclature is somewhat arbitrary because the terms could be interchanged if the interacting domains of both molecules are proteinaceous and binding/recognition is mutual.

The methodology utilizes three-dimensional representations of protein structure (e.g., Cartesian or spherical coordinate sets) to predict the necessary mutations that are required to change the amino acids in the surface of an existing binding site to bind a new ligand in place of the original ligand with a binding constant (i.e., the concentration of ligand resulting in 50% occupancy of the designed site: “affinity”) and specificity (i.e., binding of the desired “target” ligand with more favourable affinities than other “decoy” ligands that may or may not resemble the chemical structure of the target ligand) appropriate for the desired function(s) of the engineered protein(s). In addition to the redesign of known ligand-binding sites, the method can design such receptor-ligand interfaces in regions that are not necessarily known to bind ligands (de novo design of ligand-binding sites).

A process of the present invention can have the following components:

1. A three-dimensional description (e.g., Cartesian coordinate set) of the protein structure in which the ligand-binding site is (re-)designed.

2. A definition of the region where the new ligand is to bind (the “target binding site”).

3. A three-dimensional description of the target ligand, as well as any ligand degrees of freedom.

4. A description of the atomic interactions (e.g., potential function) which describes the behavior of interactions between a protein and its target ligand at the molecular level. In general, the “cost function” may include a potential function based on one or more descriptors. The cost function may also include other considerations: e.g., selection of particular amino acid residues or their statistical distribution, chemical properties built into the ligand-binding site or catalytically-active site, and quantum mechanical calculation.

5. A three-dimensional description of allowed amino acid structures used to generate mutations (amino acid “rotamer library”).

6. An algorithm that utilizes components 1-5 to predict sets of mutations in the binding site that bind the target ligand.

These components are described in detail below. In some embodiments, the invention claims novelty in:

methods for combining docking of a ligand into a protein scaffold with calculation of a stereochemically complementary surface,

the description of the target binding site (component 2),

the description of atomic interactions (component 4), and

methods for predicting mutations (component 6).

We have reduced the invention to practice by embodying the method in working computer programs (the ReceptorDesigner programs, which have been incorporated into a larger suite of computational protein design programs, known as the DEZYMER suite). Additionally, we have validated the method by experimentation and created receptors which bind trinitro-toluene (TNT), L-lactate, D-lactate, serotonin, pinacolyl methyl phosphonic acid (PMPA), or dihydroxyacetone phosphate (DHAP)/glyceraldehydes 2-phosphate (GAP) with high selectivity and affinity, using a number of different proteins as starting points. We demonstrate that these computationally predicted, engineered receptors can function as biosensors (6, 7, 12) for their new ligands, and can be incorporated into synthetic bacterial signal transduction pathways, thereby regulating gene expression in response to extracellular TNT or lactate. The use of diverse ligands and proteins proves experimentally that a high degree of control over biomolecular recognition has been established computationally. The biological and biosensing activities of the designed receptors illustrate some of the potential applications of computational design.

The process of protein design is general, and can be provided any protein structure (or model thereof) and target ligand (small molecule, protein, nucleic acid, carbohydrate, lipid, metal, or other) as input. Consequently it can be used to manipulate or introduce ligand-binding sites in any protein, for any ligand. The engineered proteins can be used either as materials ex vivo, taking advantage of the specific, high-affinity molecular recognition properties of biomolecular interactions, or can be re-introduced into living systems to function as biologically active components. The scope of potential applications of this method is therefore very large (described below), encompassing any field that takes advantage of receptor-ligand interactions.

The process is conveniently implemented as instructions for a computer system which can be comprised of a processor for calculating values from input data and otherwise manipulating data; a bus to control the flow of data between the processor and other devices, one or more input/output devices (e.g., keyboard, display, pointer, reader or writer of storage medium), and a storage medium. The instructions, data, and calculated values can be read from or written on media such as, for example, a mechanical switch or electronic valve, iron core, semiconductor RAM or ROM, magnetic or optical disk, or paper or magnetic tape. The medium can be erased, refreshed (e.g., dynamic), or permanent (e.g., static); it can be fixed or transportable.

The Receptor Design method constructs an ensemble of target ligand poses in the target ligand-binding site of the scaffold protein structure (the “Docking Zone”), and constructs an ensemble of side-chain conformations representing a set of possible mutations at each amino acid position in the target complementary surface (the “Evolving Zone”). Subsequently, degrees of freedom in the Docking and Evolving Zones are combined to identify multiple combinations of a single docked ligand pose with an associated complementary surface (mutant amino acid structure). These receptor designs are then rank-ordered using a fitness metric and a subset is submitted for experimentation (fabrication and characterization of engineered, mutant proteins). A subsequent stage can involve an iteration in which the experimental characterization of the initial set of designs is used to-construct a refined fitness metric which is then used to re-rank the designs or to produce a new set of designs that are then submitted for experimentation.

I. Components of the Calculation

Choice of Scaffold

The scaffold is a three-dimensional representation of a protein structure (a preferred embodiment is a Cartesian coordinate set specifying the position of all or a subset of atoms in the protein). This structure can be obtained using any of several methods such as, for example:

isolation from a library of experimentally-determined structures, such as the Protein Data Bank (26),

modification of such a structure by programs designed to check the plausibility of protein structures and to identify and rectify potential mistakes caused by experimental data or model fitting (27, 28),

modification by minimization of such a structure against a molecular mechanical potential, typically by conjugate gradient descent methods (29),

modification by the replacement of particular amino acid side chains by side chains of other amino acids, either naturally-occurring or non-naturally-occurring (including non-naturally-occurring side chains resulting from the coupling of a thiol-reactive group to a reactive cysteine side chain (7, 30)),

modeling by any method designed to predict protein structure from sequence, particularly homology modeling methods (31), and “ab initio folding” methodologies (32), and

construction of a “structural ensemble” containing multiple sets of coordinates, thus modeling multiple protein conformations (backbone and side chain) or any of the above modifications.

Identification of the Target Ligand-Binding Site

The target ligand-binding site is any region in the scaffold that is desired to bind the target ligand. Such a region is defined by the coordinates of the C_{α} carbon atoms in the structural model of the scaffold, or more preferably by the atoms that describe the protein “backbone” structure (any or all of amide nitrogen, amide proton, C_{α} carbon atom, C_{α} proton, carbonyl carbon, carbonyl oxygen).

For example, identification of a target ligand-binding site can be based on the experimenttally determined structure of a complex between the scaffold and one or more of its natural ligands. In this case, the atoms of the scaffold side chains that are in close contact with the ligand (the interacting atom set) are identified by measuring the linear distances between these atoms and the ligand, and selecting those amino acid atoms that are involved in hydrogen bonds, or that are in or near to van der Waals contact with the ligand. Those amino acids that have interacting atoms form the “primary complementary surface” (PCS); residues in the PCS can be truncated to alanine for target ligand docking and complementary surface generation. The PCS positions then define the target ligand binding site.

Alternatively, an entirely novel ligand-binding site can be specified ab initio by selecting a set of protein positions which can, upon mutation, plausibly provide a complementary surface for the target ligand.

Identification of the Evolving Zone and Protein Scaffold Truncation

The “Evolving Zone” (EZ) constitutes the set of residues that are allowed to mutate (“evolve”) during the course of the calculation. In the first instance, the EZ comprises the residues in the PCS (see above). An additional set of residues can be included in the EZ, comprised of the layer of amino acids that make direct contact (van der Waals interactions, hydrogen bonds) with members of the PCS. These residues interact indirectly with the ligand, forming the “secondary complementary surface” (SCS); residues in the SCS can be truncated to alanine for target ligand docking and complementary surface generation. The SCS plays an important role in stabilizing the PCS (33, 34), contributing to ligand-binding affinity and specificity, as well as protein stability. Additionally, a “tertiary complementary surface” (TCS) can be included in the EZ, comprised of residues that either form or potentially can form hydrogen bonds with residues in the SCS.

Identification of the residues in the PCS, SCS, and TCS is typically performed using an automated algorithm which analyzes residue-ligand and residue-residue distances. These automatically identified sets can also be modified by the user, generally to reflect properties of the target ligand (e.g., size, shape).

Ligand Coordinates

Three-dimensional atomic coordinates for the covalent structure or structures of the target ligand can be prepared using any of several methods such as, for example:

Isolation from a library of experimentally-determined structures, such as the Protein Data Bank (26) or the Cambridge Structural Database (35).

Modification of such a structure by addition or removal of atoms subject to commonly-accepted rules of generating molecular structure and geometry (36).

De novo modeling of the structure. This can be carried out using a software package, such as the Chem3D program of the CambridgeSoft company (http://www.cambridgesoft.com).

Initial models of molecular structure can be further refined by procedures of geometric optimization or minimization of a potential function approximating the relative free energies of various configurations and conformations of the ligand. Such a potential function can be either molecular mechanical in nature (such as the CHARMM semi-empirical potential function, or the semi-empirical potential function used in the further stages of the Receptor Design procedure), or can be quantum mechanical (such as the MM2 (37), Gaussian (http://www.gaussian.com), or MOPAC (38) molecular potentials). A covalent configuration of the target ligand is determined by specifying absolute stereochemistries for all chiral centers, and by specifying values for all bond lengths, bond angles, and non-rotatable bond dihedral angles in the molecule. Rotatable bonds are initially placed in low-energy dihedral conformations. A full explicit-hydrogen model is assumed for all molecular structures.

Description of Molecular Interactions Between the Ligand and the Protein Scaffold

The molecular interactions between the protein and its cognate ligand may be described by a potential function, the terms of which capture one or more of van der Waals interactions, hydrogen bonding, electrostatics, solvation, and internal entropies of the amino acid side chains and ligand (or all of them). Such a potential function consists of two parts: the mathematical functional forms that describe each component, and the parameters for each atom in the amino acids and ligands, that describe the magnitudes of the interactions (e.g., partial atomic charges, atomic radii, free energies of portioning between water and a non-polar reference solvent).

Ligand parameters modeling the non-bonded interactions of ligand atoms can be derived from any number of sources including, but not limited to:

Experimentally determined values of atomic radii (39), partial atomic charges (40), and hydrogen bond geometries (41).

Prediction of these parameters using any number of procedures including empirical predicttions (e.g., the Universal Force Field (UFF) procedure (42)), or quantum mechanical predictions (e.g., the MM2 package of the Chem3D program).

Similarly, the parameters for the amino acids can be taken from a variety of sources. A preferred embodiment derives the parameters from the CHARMM23 implementation of the CHARMM molecular mechanical potential function (43).

A particularly important component of a potential function, novel to a preferred embodiment of this invention, is the “hydrogen bond inventory” term. For a representative ligand L-lactate, (i) the hydroxyl group has a hydrogen bond donor and a hydrogen bond acceptor and (ii) the carboxylate group has two hydrogen bond acceptors. It has been established that in natural receptor-ligand complexes, the majority of potential hydrogen bonding groups on the ligand are satisfied either by direct contacts with the protein, or by water. Our design method therefore explicitly demands that all possible hydrogen bonding groups on a ligand be satisfied by hydrogen-bonding partners (contributed by side chain or main chain, or by explicit modeled solvent molecules). This requires specialized treatment in the design algorithms (see below).

Amino Acid Rotamer Libraries

Amino acid rotamer libraries contain descriptions (e.g., Cartesian coordinates) of all the amino acid side-chain conformations used in the calculations. Typically “rotamers” refer to the side-chain conformations corresponding to local minima (44, 45). In a preferred embodiment, we use such libraries (45) as starting points that we augment by adding in side-chain conformations that represent not only the local minima, but all energetically allowed conformations near these minima.

II. The Calculation

The calculation can take the components described above, and run the following:

1. Generation of a Docking Zone (DZ), representing all the degrees of freedom of the ligand within the target ligand binding site.

2. Generation of the Evolving Zone (EZ) by placing amino acid rotamer libraries within the EZ.

3. Minimization of the potential function over all the degrees of freedom within the EZ and DZ. This procedure produces a single docked ligand conformation, chosen from the DZ, and a single amino acid sequence, chosen from the EZ, which together correspond to the lowest value of the potential function (the global energy minimum, GEM), or near-lowest value. Together these represent the best possible design (or near best) for the design of the target ligand binding site, within the limitations presented by the description of the system (potential function, and sampling densities used to generate the amino acid rotamers and the ligand ensemble in the docked zone).

4. The GEM can be used to fabricate a single designed protein by experimentation. A preferred embodiment is to generate a set of designs that constitute “nearby” solutions to the GEM.

5. The well set is then ranked according to a fitness metric which may or may not correspond to the potential function that was used to generate the GEM (i.e., it may be another potential function or a different combination of potential functions).

Generation of the Docking Zone

Generation of the Docking Zone is preferably divided into the following:

1. Replacement of the residues in the PCS with poly-alanine or poly-glycine, thus truncating the side chains and effectively removing their identity prior to choosing the newly designed sequence.

2. Generation of all the internal degrees of freedom within the ligand (internal ligand ensemble, ILE).

3. Generation of all the allowed rotational and translational degrees of freedom of the ILE placed within the confines of the target ligand-binding site (the placed ligand ensemble, PLE).

The ILE is generated from the initial model of ligand structure by sampling of internally rotatable bond dihedral angles according to a molecular mechanical potential function, and can be performed using either a deterministic or stochastic search procedure.

Search procedures used for generation of the ILE may be:

conformational enumeration (deterministic), whereby the ensemble of ligand conformations is determined by enumeration of possibilities according to a discretization of the total allowable range of each rotatable dihedral (internal rotatable bonds have been sampled according to: hydroxyl, 360°, 3 intervals; carboxylate, 40°, 10 intervals) and

Metropolis Monte Carlo search(46) (stochastic), whereby ligand conformations are sampled according to a random walk (both the hydroxyl and the carboxylate rotatable bonds were sampled over a 360° interval, with moves being made to the internal steric interactions), using an energy-based decision criterion to accept or reject proposed conformations.

Additional ligand conformations can be obtained by sampling alternative values for bond length and angles, as well as ring puckers, alternate protonation states and partial charge sets, and low-barrier stereochemical inversions, such as at atoms with an open coordination shell.

The PLE can be generated in accordance with the following:

1. Generation of all the molecular rotations of the ILE (the rotational ligand ensemble, RLE).

2. Generation of the molecular translations of the ILE (the translational ligand ensemble, TLE).

3. Confinement of the RLE and the TLE to the target ligand-binding site.

4. Removal of all the ligands generated in stages 1-3, that make unfavorable interactions with the protein matrix surrounding the DZ.

5. Although each stage can be executed separately, for reasons of computational efficiency, a preferred embodiment is to combine all four stages into one.

6. The RLE is generated as a discrete subset of the group of rotations of a three-dimensional object. The construction of this subset of rotations is preferably performed using any of several methods such as, for example:

Using the Eulerian angle description of the rotation group (47), discrete rotations are constructed by sampling each Eulerian angle in its interval, according to a user-specified coarseness, with sampling of the second Eulerian angle weighted according to the sine of the first Eulerian angle, to avoid over-sampling near the polar regions of the rotation group.

Using the quaternion description of the rotation group (48), discrete rotations are constructed by mean square distance minimization (thus choosing a well-dispersed subset of the group of all rotations), with each member of this subset corresponding to an individual ligand rotation.

The TLE is generated by constructing a discrete set of points in the protein binding site, corresponding to potential positions of the center-of-mass of the target ligand. This discrete set of positions of the ligand center-of-mass together comprises the “docking grid” term. Generally, a cubic lattice of points is placed in the protein binding site, with user-specified rectangular lengths and lattice spacing, and the docking grid is taken as that subset of points which satisfy a user-specified minimum distance to the truncated protein scaffold. The docking grid can be modified to reflect properties of the target ligand (e.g., size, shape).

The combined RLE and TLE (docked ligand ensemble, DLE), thus constituting all possible rotations and translations of the ILE, and thus together comprising all possible compatible poses of the target ligand within the design scaffold, are constrained to the target ligand-binding site by placing a three-dimensional convex polyhedron around the target ligand-binding site and confining all or a fraction of the atoms of each member of the DLE to lie within the polyhedron. A preferred embodiment is to use a convex hull construct (49). This convex hull can be based on various objects, including the C_{α} carbon atoms of the PCS, or the van der Waals surface of the original ligand. The size of the convex hull can be adjusted by isometric expansions or contractions.

Generation of the Evolving Zone

Generation of the Evolving Zone involves placement of amino acid rotamer libraries at each of the residue positions in the EZ, and removing those members of the rotamer library so placed, that form interactions with the surrounding protein matrix, which exceed some threshold value (defined by the user) of the potential function. The rotamer libraries can contain representtations of amino acids in various combinations:

mutation to any of the twenty naturally-occurring amino acids.

mutation to any of a subset of the naturally-occurring amino acids. Typical subsets of amino acids constructed include, but are not limited to:

all amino acids with hydrophobic side chains.

all amino acids with hydrophilic side chains.

all amino acids except proline, cysteine, and glycine.

mutation to any set of amino acids, including any or all of the naturally-occurring amino acids, and also including a set of non-naturally-occurring amino acids, including, but not limited to, amino acids resulting from the reaction of cysteine with a thiol-reactive group.

sampling side-chain conformation, with preservation of amino acid identity (i.e., allow the structure of a single amino acid side chain to vary in the course of the calculation).

preservation of amino acid identity and side-chain conformation (i.e., a single fixed structure).

Typical combinations of allowed degrees of freedom for the PCS, SCS, and TCS include, but are not limited to:

PCS allowed to mutate to all naturally-occurring amino acids; SCS, TCS fixed.

PCS allowed to mutate to all naturally-occurring amino acids; SCS allowed to alter side-chain conformation; TCS fixed.

PCS, SCS allowed to mutate to all naturally-occurring amino acids; TCS fixed.

PCS, SCS allowed to mutate to all naturally-occurring amino acids; TCS allowed to alter side-chain conformation.

The endpoint of a receptor design calculation consists of a set of individual predicted modes of ligand binding, each associated with a set of mutations to the design scaffold predicted to provide a complementary protein surface to facilitate ligand binding. Preferred are two distinct methods for the discovery of these individual ligand pose-protein sequence combinations:

(i) the method of enumeration of complementary protein surfaces for a discrete and representative subset of the DLE, thus approximating all possible poses of the ligand in the target binding site or

(ii) the method of simultaneous ligand-protein optimization, whereby the DLE (all ligand degrees of freedom) is treated as a super-rotamer, akin to the amino acid side-chain rotamer degrees of freedom at the positions of the protein.

These two sequence design methods are described below; the method of representative subset enumeration is a preferred embodiment for sequence design.

Sequence Design: 1. The Representative Subset Enumeration Approach

Given the ligand structures in the DZ (together constituting the DLE), and amino acid side-chain structures in the EZ, each generated in the stages described above, the global energy minimum (or approximation thereof) is identified in two stages:

1. Generation of compatible ligand poses (CLIPs).

2. Sequence optimization (the INTERFACE procedure) in the EZ for each CLIP. A CLIP is a single ligand conformation (“pose”) docked into the target ligand-binding site; together the CLIPs constitute a representative subset of all DLE members. For each such conformation, a design calculation is carried out in which a single EZ sequence corresponding to the GEM or aGEM (approximate global energy minimum) is identified in the INTERFACE procedure; these GEM (aGEM) values are local to the CLIP under consideration (the CLIP GEM, cGEM). This approach is essentially an enumeration of the EZ GEMs (aGEMS) for all the CLIPs. This representative enumeration is a preferred embodiment of the sequence design algorithm, because it allows the critical and specialized hydrogen-bond inventory (as well as other) constraints to be applied to the design process (see below).

Generation of CLIPs

In a typical calculation, the size of the DLE is too large for enumeration of each member in the ensemble by the INTERFACE procedure in a finite time. Consequently, a representative subset is chosen, the CLIPs (in the limit, the set of CLIPs is the same as the DLE). The CLIPs are chosen by rank-ordering the DLE according to the interaction energy between each ensemble member and the scaffold in the truncated target site form (the scaffold interaction energy, Eₛ). The Eₛ term consists of van der Waals, hydrogen bonding and electrostatics components, each of which can either be included or omitted, as the user desires. For a given form of Eₛ, the DLE can be rank-ordered according to Eₛ itself, or the absolute value of Eₛ. In the former case, the top-ranked DLE member represents the ligand pose that has the most favorable interactions with the truncated design scaffold; in the latter case, the top-ranked member corresponds to the ligand that has the least interactions (favorable or otherwise) with the scaffold. Both rankings are equally valid. In addition, a differentness metric can be applied to members of the DLE, in order to generate a set of CLIPs that together represents all possible compatible ligand poses. In its simplest implementation, the differentness metric takes the form of insisting that each member of the TLE (the “docking grid”) contribute a docked ligand pose to the set of CLIPs. In more complex implementations, the DLE members can be assayed for degree of pairwise overlap, with “overly similar” DLE pairs prevented from simultaneously existing in the ensemble of CLIPs.

The INTERFACE Procedure

The INTERFACE procedure identifies protein side-chain sequences and structures of the binding-site residues which are determined to be compatible both with individual ligand poses and the protein scaffold. In practice, this is performed by finding protein sequences and structures which minimize a semi-empirical potential function describing the interactions between the components of the biomolecular system (protein and ligand), with treatment of the ligand and its interactions as a privileged component. The INTERFACE procedure employs a cycle between a computational search strategy to identify protein sequences predicted to minimize the potential of the entire biomolecular system, and specialized sequence design algorithms (the INCREDIBLE algorithms) to identify and eliminate particular side-chain structures incompatible with a well-formed interface between protein and ligand, for example, those side chains whose presence results in unsatisfied ligand hydrogen-bonding potential, or the disruption of the lock-and-key fit between protein and ligand.

The sequence design algorithms can be any one that has been developed for sequence optimization (these can be stochastic or deterministic) which include, but are not limited to:

Simulated Annealing algorithms for sequence design (50) (stochastic).

Monte Carlo search algorithms for sequence design (51) (stochastic).

Genetic Algorithms for sequence designs (52) (stochastic).

Dead-end elimination (DEE) algorithms for sequence design (16, 53) (deterministic).

FASTER algorithms (54) (deterministic/stochastic).

Enumeration algorithms for sequence design (55) (deterministic).

In a preferred embodiment, we use a combination of DEE and FASTER algorithms, which together with the INCREDIBLE algorithms, designs a highly complementary surface to an individual CLIP pose.

The INCREDIBLE Algorithm

The INCREDIBLE (INCompatible Rotamer Elimination for the Design of Interfaces and Binding of Ligands), algorithms captures critical aspects of molecular recognition, such as the lock-and-key steric complementarity between protein and ligand (56), and the satisfaction of the hydrogen bond inventory of the ligand (18), which are deemed to be more important to successful interface design than is the value of the overall molecular potential (which can include interactions distal from the ligand). Each of the INCREDIBLE algorithms employed in a calculation is applied iteratively as the sequence design algorithm converges, in stages, towards an energy minimum of the entire biomolecular potential of the system. The INCREDIBLE algorithms function to drive the designed protein sequence towards solutions which optimize characteristics of the immediate receptor-ligand interface, as opposed to those designed sequences many of whose favorable interactions are not between protein and ligand. Any quantitative characteristic of the receptor-ligand interface can be employed to drive an INCREDIBLE algorithm, although there are two preferred embodiments:

1. The “hydrogen bond inventory” of the ligand. In this INCREDIBLE algorithm implementation, the sequence design algorithm is guided into any subset of sequence space which can be determined to be that most likely to completely or maximally satisfy the “hydrogen bond inventory” of the target ligand, i.e., all ligand hydrogen bond donors and acceptors. In this manner, designed sequences which form some favorable interactions but fail to satisfy the hydrogen bonding capacity of the ligand (a critical component of a well-formed interface), are iteratively pruned from the available sequence space, thus ensuring ligand hydrogen bond inventory satisfaction, regardless of the other components of the overall biomolecular potential function. In the standard implementation of this INCREDIBLE algorithm, if at any point during the sequence optimization, it can be determined that all remaining side chains which satisfy a particular ligand hydrogen bond arise from the same protein position, then all non-hydrogen-bonding side chains at this position are eliminated from the sequence space. This ensures that the designed protein sequence satisfies this element of the ligand hydrogen bond inventory.

2. The elimination of cavities from the designed receptor-ligand interface (the “binding surface inventory”). The implementation of this INCREDIBLE algorithm is similar to that for the ligand hydrogen bond inventory. If, at any point in the complementary surface optimization, it can be determined that a particular and substantive portion of the ligand binding surface can be in close association (“binding surface satisfaction”) with only protein side chains arising from a single residue position, then all side chains which do not satisfy this binding surface (“cavity-forming” side chains) at this position are eliminated.

Sequence Design: 2. the DLE Super-Rotamer Method

In an alternative to the method of CLIP representative subset generation, the problems of ligand pose placement and protein sequence design can be combined, with the resulting GEM or aGEM thus constituting a ligand pose and an associated protein complementary surface, which is deemed to be the best possible (or near best) design for the ligand binding site, as determined by the value of the design potential for the ligand-protein system. The DLE super-rotamer method is incompatible with the INCREDIBLE algorithms, however, which are an important driving force for optimization of the immediate receptor-ligand interface. It is for this reason that the CLIP representative subset generation method is a preferred embodiment for generation of the initial family of receptor-ligand designed interfaces.

Generation of Well Sets

Although the sequences corresponding to the GEM or aGEM of the system are invaluable reference points in the design procedure, it is typically necessary to identify other sequences that are closely related either in sequence space (e.g., single point mutations or combinations thereof), or in energy space (e.g., within an interval ΔE_{well} of the GEM or aGEM of the entire system); such sequences are designated by the “well set” term. The generation of well sets has two functions: a) it provides a set of plausible designs for empirical evaluation which mitigates prediction inaccuracies and b) it allows potential functions other than the one used to generate the GEM (aGEM) or the well set to be used (see description of the LORD procedure below). Of particular value is to generate a well set that falls within ΔEM_{well} of the GEM or aGEM, and then to rank-order these according to some evaluation criteria other than the original potential function.

Well sets can be generated by the following:

1. Use all the cGEMs as a well set.

2. Stochastic or deterministic generation of well sets from the GEM, aGEM, or from cGEMs, using the OVERLORD procedure (Optimize, Vary, & Explore Related sequences with the LORD procedure) described below.

Ranking Wells: the LORD Procedure

Well members can be ranked according to the potential function used in the calculation. However, a more typical ranking method is to use descriptors that are more sophisticated than the potential function used to generate the well members in the first place. This is performed by the LORD (Linear Optimization of Ranking Descriptors) procedure, using ranking descriptors that are intended to be a more realistic evaluation of the quality of a ligand-protein interface, and can differ greatly in functional form (typically not pairwise-decomposable, as is the design potential) and ease of computation (typically more time consuming) from the semi-empirical design potential. Ranking descriptors employed in the LORD procedure may include, but are not limited to:

value of the semi-empirical design potential restricted to the immediate receptor-ligand interface

value of the semi-empirical design potential for the entire designed protein

number of unsatisfied hydrogen-bonding atoms in the ligand

number of unsatisfied hydrogen-bonding atoms in the PCS

exposed solvent-accessible surface area (SASA) of the ligand

total volume of any cavities in the ligand-protein interface

total enthalpy of all hydrogen bonds between protein and ligand

steric complementarity of ligand and protein, as determined by:

total van der Waals interactions

complementary interaction surface area (57)

Voronoi tessellation (58)

There are two forms of the LORD procedure:

1. Protein sequences are chosen from the set of all well members, which simultaneously score well according to each ranking descriptor, to a user-specified extent for each ranking descriptor (either by restricting the analysis to those well members which score in some top fraction for each ranking descriptor, or which have a value of a ranking descriptor less than some absolute value, typically in the case of the unsatisfied hydrogen bond descriptor). All well members which thus perform satisfactorily well according to every ranking descriptor are finally rank-ordered according to a user-specified ranking descriptor deemed to be the most indicative of the quality of the receptor-ligand interface, with this rank-ordered list being submitted to further analysis.

2. Any combination (linear or otherwise) of existing ranking descriptors constitutes a further ranking descriptor, which captures aspects of its component descriptors. This is most useful when a large database of designed receptors have been characterized both in silico and in vitro. In this instance, a quantitative structure-activity relationship (QSAR) can be constructed to postdict the experimentally determined performance of each receptor (ligand binding affinity, ligand binding specificity, receptor stability) in terms of the ranking descriptors computed for that receptor and receptor-ligand interface. In this manner, a novel ranking descriptor of maximal correlation is constructed against the experimental data. This “semi-empirical” ranking descriptor can then be used in further design of receptors for the same ligand, similar ligands, or even structurally and chemically diverse ligands.

Ranking Descriptors not Based on the Semi-Empirical Force Field

Many ranking descriptors are obtained by application of the semi-empirical design potential (or particular components) to a subset of the system, particularly the receptor-ligand interface. Some, however, are of a different nature:

The solvent-accessible area (SASA) of the target ligand within a designed interface can be computed by the Connelly surface area algorithm with a probe radius of 1.4 Å. The SASA of the target ligand is computed within the designed well member complementary surface, using a full hydrogen model.

A ranking descriptor which describes cavities between protein and ligand is also commonly employed. A cubic lattice of grid points of user-specified rectangular lengths and grid spacing is placed around the ligand in the well member binding site. Each of these points is queried for distance to ligand, protein, and bulk solvent. Those points which are sufficiently distant from protein and ligand to represent electron density coverage of either (typically set at 1 Å), but simultaneously sufficiently close to prevent explicit solvent molecule entry (typically set at 1.5 Å), are deemed to constitute a cavity between protein and ligand. This set of “cavity points” is converted to a “cavity volume” which is used as a ranking descriptor.

An independent estimator of ligand affinity can be used as a ranking descriptor. This can take the form of an external software package, e.g., a quantum mechanical program with ligand affinity estimation capability.

When the designed complementary surface is intended to be catalytically active (i.e., an enzyme design calculation), any estimator of reactivity of the ligand (substrate)-complementary surface pair can be employed as a ranking descriptor. This can consist of any prediction of pKₐ or electron localization for predicted active set residues, or any external software package for the modeling of protein-substrate reactivity.

Generation of Wells: the OVERLORD Procedure

This “well exploration” can be performed by any computational search strategy (deterministic or stochastic), with a preference for Monte Carlo-based stochastic search techniques (51), or a search algorithm based on either the DEE (24, 59) or the FASTER computational search strategy (54):

In a typical Monte Carlo stochastic search, random steps in sequence space (typically point mutations) are taken around GEMs or aGEMs to generate an ensemble of well member sequences. Moves in sequence space are typically accepted according to a probability which decreases according to the size of the potential energy increase. Multiple, independent random walks can be initiated around a given GEM or aGEM, with the resulting sequences wells being collated. Well member sequences can additionally be constrained to lie within a fixed ΔE_{well} potential energy difference from the initial GEM.

The DEE algorithms (24, 59) can also be used with a fixed, positive value of ΔE_{well} to eliminate individual rotamers which can provably not be a member of any sequence within ΔE_{well} of the GEM. Any remaining sequence space can be explored by enumeration or a tree search method to construct well members.

A modification of the FASTER algorithms (54) which combined perturbation, relaxation, and random mutagenesis can be used to construct well members. In this search strategy, the initial GEM sequence is subjected to iterative rounds of random mutagenesis (a user-specified number of point mutants), followed by a standard implementation of the FASTER algorithms (typically batch relaxation or single-residue perturbation/batch relaxation) to optimize the remainder of the sequence not the subject of the random mutagenesis. Multiple, independent trajectories can be taken away from the initial GEM, with the results being collated.

Quantitative Structure-Activity Relationships (QSARs)

QSAR construction is typically performed by single variable, linear regression to optimize coefficients of the separate ranking descriptors (independent variables) to maximize the correlation (R-value) of the experimentally determined receptor performance (e.g., ligand binding affinity, catalytic rate, other biochemical activities).

### FIELDS OF APPLICATION

As demonstrated, the computational design methodology is general, and can be given any protein structure (or model thereof) and target ligand (small molecule, protein, nucleic acid, carbohydrate, lipid, metal, or other) as input. Consequently it can be used to manipulate or introduce ligand-binding sites in any protein, for any ligand. The engineered proteins can be used either as materials ex vivo, taking advantage of the specific, high-affinity molecular recognition properties of biomolecular interactions, or can be re-introduced into an organism to function as in vivo biologically active components.

Nucleic acid encoding protein(s) designed by the invention can be introduced by gene transfection, viral infection, or recombination with an endogenous gene. It can interact with an endogenous pathway (e.g., receptor) or a pathway with one or more exogenous components (e.g., kinase, phosphatase, other enzyme, channel or transporter). The organism may be microbial (e.g., archaebacterium, eubacterium, fungus, virus), animal, or plant. A DNA or RNA vector comprised of a nucleotide sequence encoding the protein(s) and one or more regulatory regions (e.g., constitutive or inducible promoter; other regions which regulate transcription, translation, or replication) may be used to transfer and/or to express sequences.

The protein may be chemically synthesized, in vitro transcribed/translated (e.g., cell-free systems, reticulocyte lysate), or expressed in a cultured cell or organism. One or more non-natural residues may be substituted for an amino acid residue of the protein by chemical synthesis or elongation with an artificially charged transfer RNA. One or more non-natural side chains may also be incorporated into the protein in this manner. Protein may also be post-translationally modified. Therefore, the chemical properties of a side chain or its geometric positioning in the protein may be determined by a structure other than the 20 natural amino acid residues. The protein may be comprised of the mature amino acid sequence (see Tables 1, 3 and 5) as well as other protein domains (e.g., a signal peptide which causes secretion, another cell localization signal, an anchor peptide which is membrane inserted, an affinity peptide for purification). Synthetic peptide cleavage signals may be inserted between such domains to produce mature protein by proteolysis. Protein may be purified by biochemical procedures known in the art: centrifugation, chromatography (e.g., affinity, ion exchange, gel sizing, hydrophobic/hydrophilic interaction), electrophoresis, and precipitation.

The protein designs obtained by the invention may be used as a library of amino acid sequences prior to confirmation of binding to ligand or an analog thereof. For example, the library may be used with or without other sequences in a gene shuffling or directed/random evolution process to provide improved proteins whose binding activity is then confirmed. The high efficiency of the invention in designing protein with binding activity may provide one or more potential mutants which can be further manipulated without experimentally confirming that they bind ligand. Alternatively, confirmation of binding may be performed with an analog of the ligand which is bound (e.g., PMPA in Example 2) or the reactive substrate or product of an enzyme (e.g., DHAP and GAP in Example 3).

The protein may be designed with more than 10, more than 15, more than 20, more than 25, or more than 30 changes in the amino acid sequence as compared to the starting protein for which a structure has been determined or is predicted. Thus, the structure of a protein may be used to predict the structure of a mutant or analog thereof which is the basis for a new protein design. The ligand may bind protein with at least micromolar, at least nanomolar, or at least picomolar affinity. For a protein with catalytic activity, a rate enhancement of at least 10³-fold, at least 10⁴-fold, at least 10⁵-fold, at least 10⁶-fold, at least 10⁷-fold, at least 10⁸-fold, or at least 10⁹-fold over the uncatalyzed reaction is preferred.

The scope of potential applications of this method is large, encompassing any field that takes advantage of receptor-ligand interactions, including, but not limited to:

The construction of biosensors (ex vivo or in vivo), in which the (re-)designed protein functions as a molecular recognition element for an analyte and is coupled to a signal transduction mechanism that couples ligand binding to a readout signal that can be utilized in a detector (6, 67, 68).

Affinity purification reagent (ex vivo), in which the (re-)designed protein functions as a molecular recognition element that preferentially binds a molecule in a mixture.

Chiral purifications (ex vivo), in which the (re-)designed protein functions as a molecular recognition element that preferentially binds one stereoisomer over others (10, 69).

Synthetic signal transduction pathways (in vivo) in which (re-)designed receptors mediate a biochemical response to a ligand (70) (agonist or antagonist).

Synthetic genetic circuits (in vivo) in which (re-)designed proteins mediate the ligand-dependent action of a genetic control element (1, 71, 72) (including but not limited to repressor or activator proteins).

(Re-)Design of allosteric regulator elements in enzymes, receptors, or DNA-binding protein, in which the binding site is structurally, thermodynamically and kinetically coupled to another site (or multiple other sites) such that binding of a ligand at the (re-)designed site alters the activity at the other site(s).

Synthetically controlled metabolic pathways (in vivo), in which an enzyme with an engineered allosteric control element is used to control the flux of metabolites through a pathway.

Enzyme redesign to alter the binding specificity of a known enzyme active site.

Enzyme design in which a new catalytically active site is constructed (73).

The range of ligands that can be addressed by the computational design algorithms described here include, but are not limited to:

Toxins, including but not limited to:

Chemical warfare agents

Biological warfare agents

Industrial pollutants

Pesticides & herbicides

Carcinogens

Neurotoxins

Explosives

Metabolites

Drugs and drug precursors

Neurotransmitters

Disease state indicators

Chiral fine chemicals

Precursors and components in the stages of a (bio-)chemical synthesis

The range of proteins that can be used as scaffolds for the computational design algorithms described here include, but are not limited to:

The family of bacterial periplasmic binding proteins (PBPs), including but not limited to the Gram negative receptors for amino acids, carbohydrates, cations, anions, and vitamins.

The superfamily of proteins containing the PBPs, including but not limited to the eukaryotic glutamate receptors, transcription factors including lac, enzymes such as cyclohexadienyl dehydratase (74).

The superfamily of nuclear metabolite receptors, including but not limited to receptors for hormones, vitamins, xenobiotics, and fatty acids (75).

Proteins with multiple, allosterically-coupled, binding sites.

Antibodies (76).

Beta-clamshell proteins, such as olfactory proteins (77).

The family of cytoplasmic, antiparallel 5-barrel ligand-binding proteins, such as the fatty acid binding proteins (78).

Proteins which function as members of enzymatic pathways, whereby redesign of a binding site allows for the creation of pathways with novel functionalities.

Biosensors

At the molecular level, biosensors combine molecular recognition with transduction of a ligand-binding into a detectable physical signal that can be utilized in the construction of a device for the detection of the analyte (6). Biosensors can utilize any protein that binds a ligand including, but not limited to enzymes, receptors or antibodies. Signal transduction can take place entirely in vitro by integrating the molecular recognition element into a physical device (67), or it can be cell-based (68) in which the molecular recognition element controls a biochemical or genetic response. The computational design process described here can be used to construct the molecular recognition element in such biosensors. An advantage of the invention is that by suitable attachment of a reporter group in the hinge of PBP (or an allosteric movement of the receptor in response to binding of ligand), no addition of exotic reagents is need to generate a signal.

An example of the utility of the computational design methodology is afforded by the redesign of the PBPs to bind target ligands unrelated in structure to the natural ligand. The PBPs have been engineered to couple ligand-binding events to changes in fluorescence (7, 34, 79, 80) or redox activity (81), by coupling fluorophores or redox reporter groups respectively at locations where these reporter groups are sensitive to ligand-mediated hinge-bending motions that typify this protein superfamily. These engineered proteins therefore function as reagentless optical or bioelectronic sensors for the ligands to which they bind. This reagentless coupling mechanism is maintained even upon drastic redesign-of the ligand-binding sites (11, 34). Consequently, the computational design methodology described here enables families of biosensors to be engineered for any ligand that can be accommodated in such PBPs.

Potential applications for engineered biosensor proteins include but are not limited to:

Food processing management.

Detection of pollutants and toxins as an intial stage in bioremediation.

Detection of explosives, chemical threats, and biological threats for purposes of homeland security and weapons inspection.

Detection of disease state indicators and metabolite concentration determination for

Real-time health monitoring.

Basic biomedical research, such as the detection of particular metabolites (metabolomics) or signal-transduction intermediates.

Drug detection and drug concentration determination for purposes of:

Monitoring drug administration regimens.

Detection of banned substances.

Determination of individual pharmacokinetic response.

Detection of final product and precursors of the synthesis of:

Pharmaceuticals.

Fine chemicals.

Detection of enantiomers and diastereomers, particularly of pharmaceuticals and fine chemicals, which proves difficult by traditional chiral separation techniques.

Affinity Purification

Proteins that have been engineered by the computational design methodology described here to bind preferentially a particular molecule can be used to selectively purify or deplete that molecule from a complex mixture by affinity chromatography. In this method, the engineered protein is immobilized on a solid support. Upon exposure of this derivatized support to a complex mixture, the molecule of interest will be selectively adsorbed onto the matrix. Such a matrix can be used either in batch purification (matrix is mixed with mixture, and allowed to settle out) or in column chromatography (matrix is confined, and mixture is flowed through). This affinity chromatography methodology can be used to purify molecules from a complex mixture such as multiple products obtained in a chemical synthesis. The methodology can also be used in detoxification using the matrix to deplete a toxic molecule from a mixture. Solutions of interest for such detoxifications include, but are not limited to, drinking water or blood.

Signal Transduction Pathways

The control of cellular physiology and gene transcription in response to extracellular or intracellular signals is a fundamental property of living systems. Such responses are mediated by complex pathways that are initiated and regulated by ligand binding to receptor. An illustration of this is afforded by the demonstration that redesigned PBPs can control signal transduction pathways that respond to the target ligands upon re-introduction into E. coli (see below).

Such synthetic signal transduction pathways can be used to engineer cells, tissues, and whole organisms in principle to link any input to any output. Applications include, but are not limited to:

Cell-based biosensors by coupling the input to changes in an electromagnetic (e.g., current, voltage, frequency) or optical (e.g., intensity, wavelength, polarization) signal readable as a detectable output (e.g., colored light, fluorescence).

Cell-based bioremediation by coupling the input to production of enzymes to degrade the target ligand(s).

The engineering of smart therapeutic cells, by coupling the input to the production of repair enzymes, or agents that kill pathogens or cancerous cells, or to the secretion of a therapeutic molecule, such as a small organic molecule (e.g., drug), hormone (e.g., insulin), or immuno-regulatory molecule (e.g., cytokine).

Induction of differentiation such as the production of fruiting bodies in response to an external ligand.

Chiral Purification

A special case of affinity purifications described above is that of chiral purifications. Many molecules possess asymmetric centers. Consequently, molecules can exist in multiple, structurally distinct forms (stereoisomers). This asymmetry is of particular importance in living systems, since proteins typically interact with one stereoisomer only. Consequently, for many drugs, only one stereoisomer (the “eutomer”) exhibits the desired pharmacological activity, whereas the other stereoisomer(s) (the “distomer(s)”) are either inactive, or associated with side-effects (10). Chiral purification of drugs is therefore of great importance for safe administration, and nowadays is mandated by the U.S. Food and Drug Administration (82). The importance of chirality applies not only to drugs, but to most complex chemical materials. The computational design technique can be used to design proteins that bind one stereoisomer preferentially over another.

Such chiral purifications are illustrated by design GBP.G1, a GBP variant designed to bind L-lactate (Table 1). This designed receptor differentiates between L- and D-lactate (Table 2). Separate columns were prepared with wild-type GBP and GBP.G1 respectively covalently coupled to the resin. A racemic mixture of L- and D-lactate was applied to each column, and the eluate assayed optically for lactate. The designed receptor cleanly separates the two enantiomers, whereas the wild-type protein does not.

Design of Enzymes

With the input of a transition state model for a particular catalytic conversion as the target ligand, a Receptor Design calculation allows for the construction of proteins predicted by the “transition state stabilization” theory of catalysis (18) to function as enzymes (e.g., oxido-redutases which catalyze oxidation-reduction reactions, transferases which catalyze transfer of functional groups, hydrolases which catalyze hydrolysis reactions, lyases which catalyze additions to a double bond, isomerases which catalyze isomerization reactions, and ligases which catalyze formation of bonds with ATP cleavage) catalyzing that particular molecular conversion. Kinetics of the enzyme, its substrate and/or cofactor specificity, and inhibition can be changed. More generally, the Receptor Design algorithm can be used in conjunction with other computational techniques, such as the “site search” method of geometric optimization (85) or a quantum mechanical design methodology. After positioning of the catalytic active site residues by one of these methods, the Receptor Design algorithm can be employed to design the remainder of the complementary surface in the active site. Optionally, directed or random mutagenesis methods (i.e., site-directed mutagenesis, error-prone polymerase, gene shuffling, directed evolution) may be used after design of the ligand-binding site and/or catalytically-active site to improve binding affinity, catalytic rate, enzyme turnover, protein stability, or a combination thereof.

### EXAMPLE 1

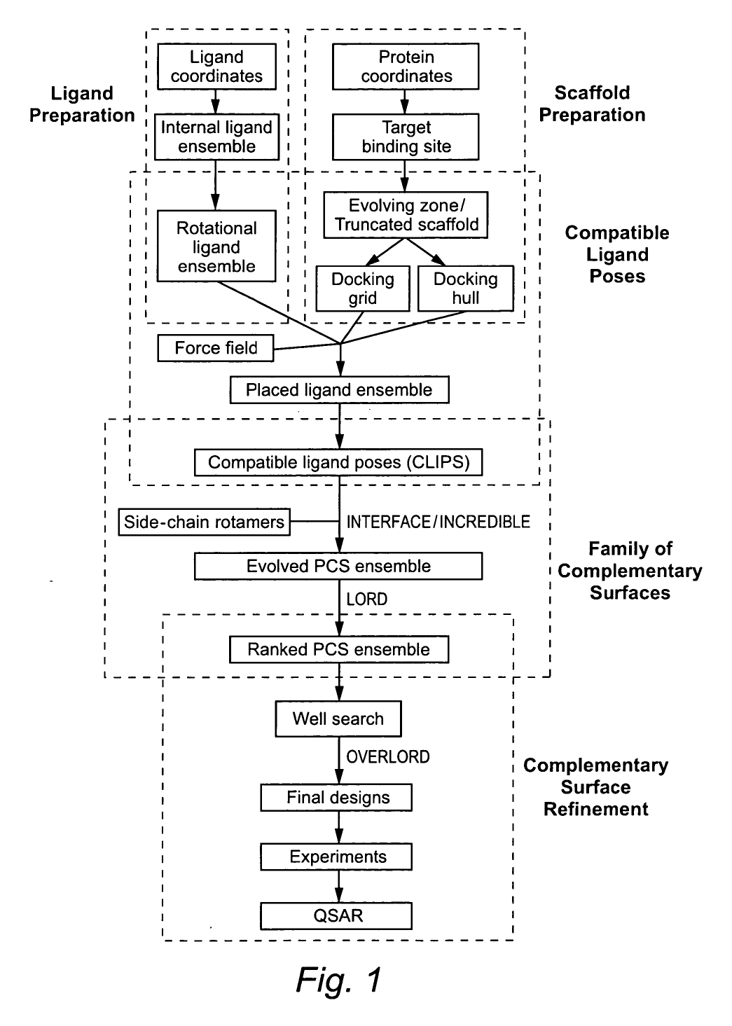

The computational design method described above has been reduced to practice in a specific embodiment (FIG. 1) in operational computer programs (ReceptorDesigner programs that form a component of the DEZYMER suite) and experimental validation of designs gene-rated by the ReceptorDesigner programs.

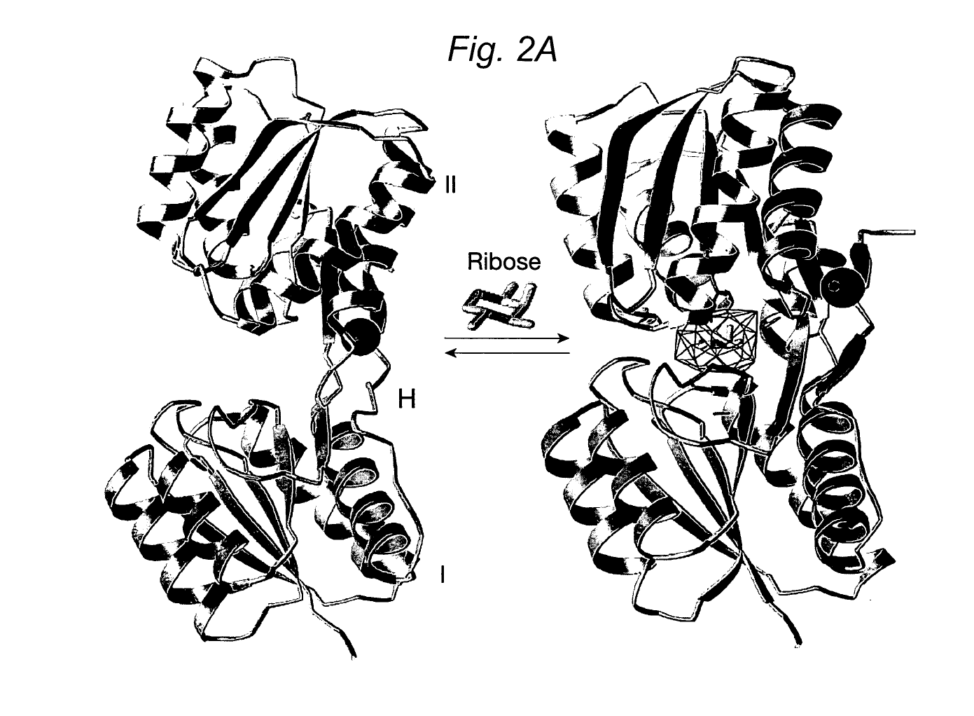

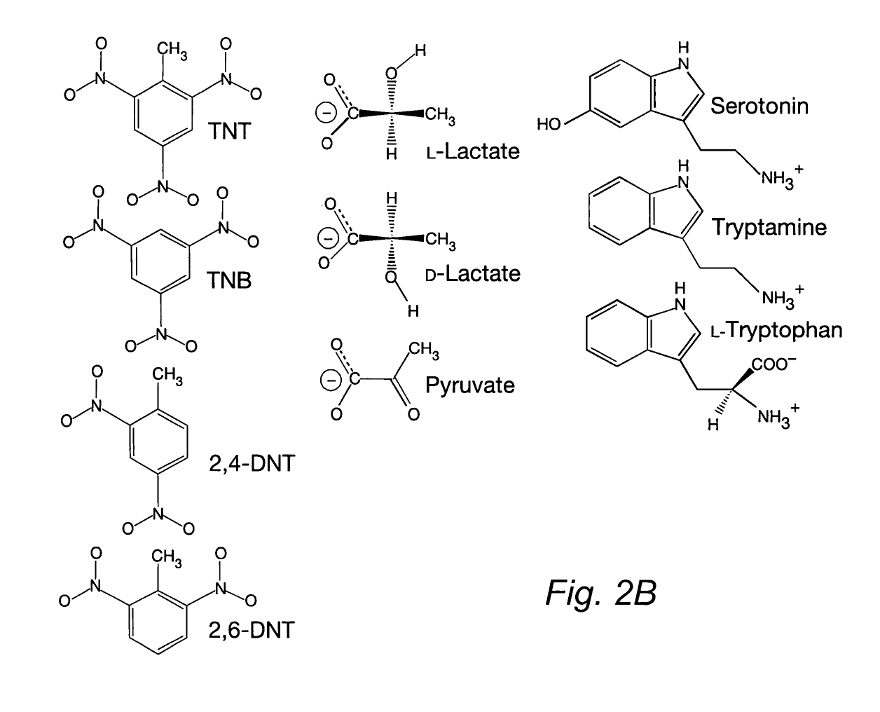

The Receptor Design procedure was used to engineer TNT, L-lactate, D-lactate, or serotonin binding sites in place of the wild-type sugar or amino acid ligands of five members of the Escherichia coli periplasmic binding protein (PBP) superfamily (60), using the high-resolution three-dimensional structures of the closed conformation of these proteins complexed with their wild-type ligand as starting points for the calculation (FIG. 2A): glucose-binding protein (GBP) (61), ribose-binding protein (RBP) (62), arabinose-binding protein (ABP) (63), glutamine-binding protein (QBP) (64), and histidine-binding protein (HBP) (65); the PDB database lists the structures and wild-type amino acid sequences as 2GBP (SEQ ID NO: 1), 2DRI (SEQ ID NO:2), 1ABE (SEQ ID NO:3), 1WDN (SEQ ID NO:4), and 1HSL (SEQ ID NO:5) respectively. These periplasmic proteins are synthesized as precursors consisting of a signal peptide and the mature amino acid sequence provided herein. The variation in structure and sequence (60) of these proteins presents distinct starting points for the design calculations. The three target ligands selected for this study bear little resemblance to the wild-type, cognate ligands of the chosen PBPs, are chemically distinct from each other, and in one case (TNT) represent a non-natural molecule. The designs therefore explore critical parameters of molecular recognition, including molecular shape, chirality, functional groups (hydrogen bonding: nitro (acceptor), hydroxyl (donor and acceptor), carboxylate (acceptor); molecular surface: polar, aliphatic, aromatic), internal flexibility (TNT<L,D-lactate<serotonin), charge (TNT: neutral; L,D-lactate: anionic; serotonin: cationic), and water solubility (TNT<serotonin<L,D-lactate).

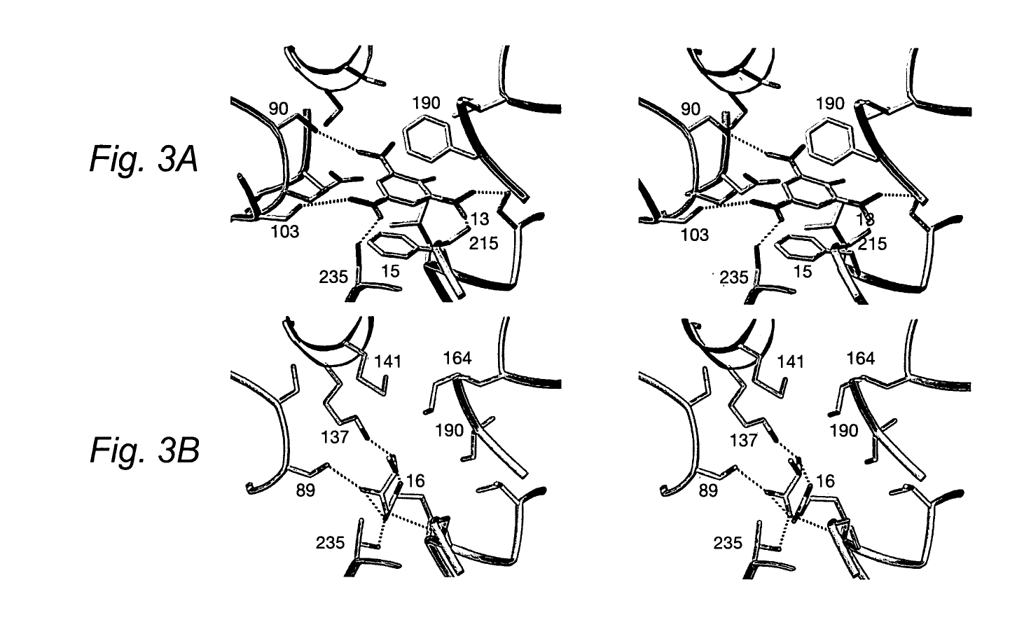

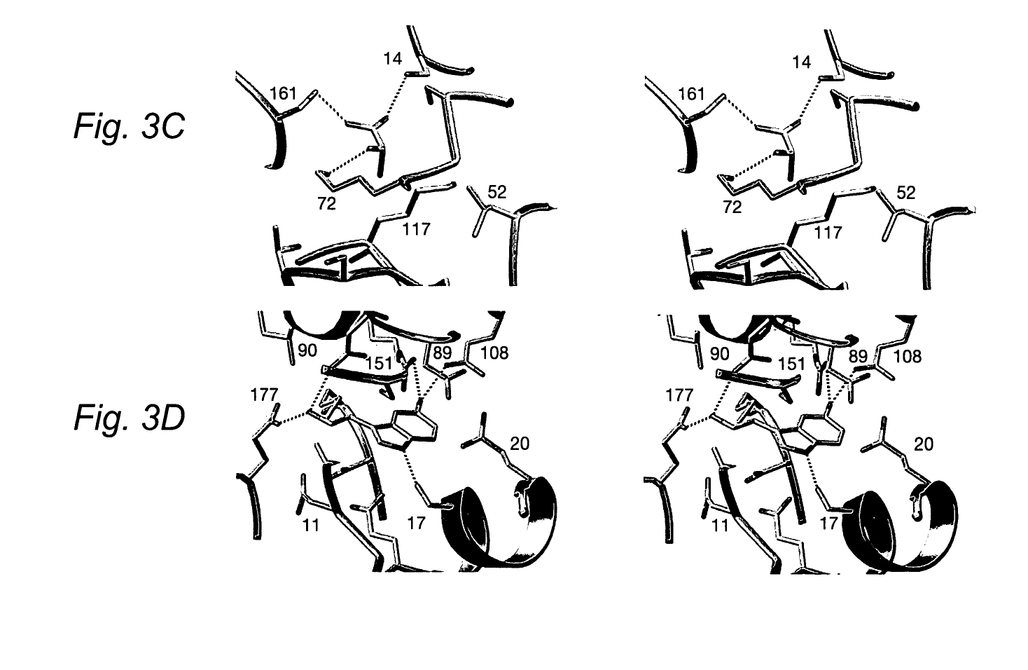

Complementary surfaces were designed for TNT in RBP, ABP, and HBP; for L-lactate in ABP, GBP, RBP, HBP, and QBP; for D-lactate in GBP; and for serotonin in ABP (FIG. 3; Table 1). The designed surfaces are electrically neutral for TNT, positively charged for lactate, and negatively charged for serotonin. Hydrophobic groups of all three target ligands interact primarily with aliphatic side chains, although several examples of aromatic interactions are seen (TNT.A1, TNT.H1, L-Lac.G1, D-Lac.G1, D-Lac.G2). In one instance, an example of dual aromatic stacking was obtained (TNT.R3). In all cases, the hydrogen-bonding potential (donor, acceptor) of the functional groups on the ligand is largely satisfied.

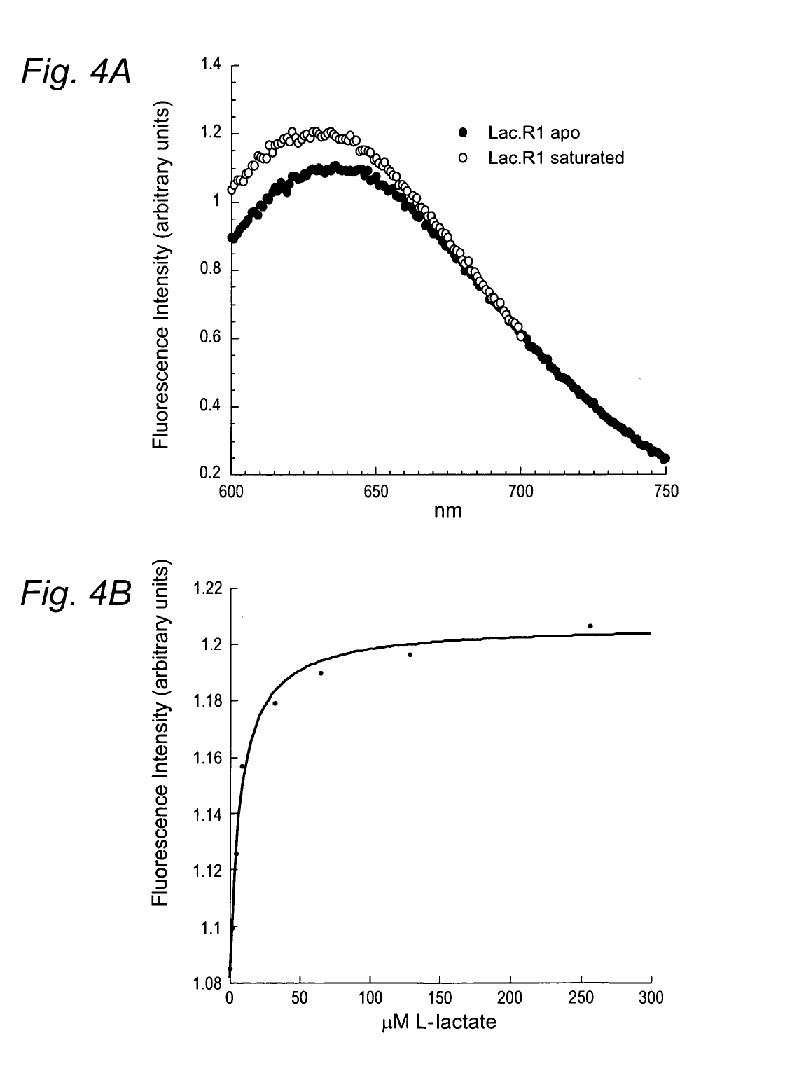

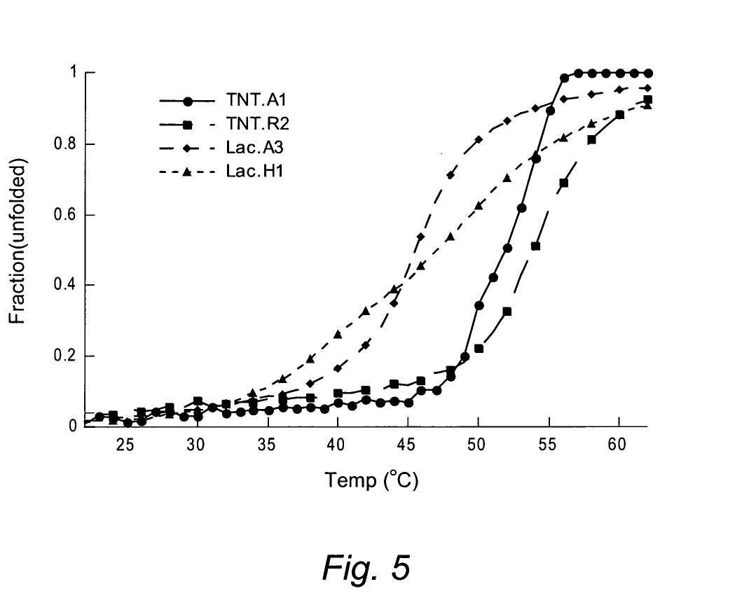

Twenty designs predicted by the automated design procedure were selected for experimental characterization (Table 1). The predicted mutations (ranging from five to seventeen amino acid changes) were constructed by PCR mutagenesis of the wild-type receptor scaffold genes (7). Proteins were over-expressed, purified, and modified with thiol-reactive styryl dyes conjugated to cysteine residues introduced by mutation at locations where the fluorescence emission intensity of the dye responds to a ligand-mediated hinge-bending motion of the receptor (7). Ligand-binding affinities (Table 2) were determined by titration, monitoring ligand-dependent changes in fluorescence emission intensity that were fit to single-site binding isotherms (7) (FIG. 4). In all cases, wild-type receptors show no change in fluorescence intensity upon addition of target ligands. Conversely, the mutant receptors respond only to target and not wild-type ligands. A wide range of affinities is observed, down to the nanomolar level (TNT.R3). To probe the specificity of interaction, the affinities of a number of closely related ligands (FIG. 2B) were also determined (Table 2). The thermostabilities of a representative subset of aporeceptors (FIG. 5) showed that cooperative folding transitions are retained with a slight loss of stability relative to the wild-type proteins.

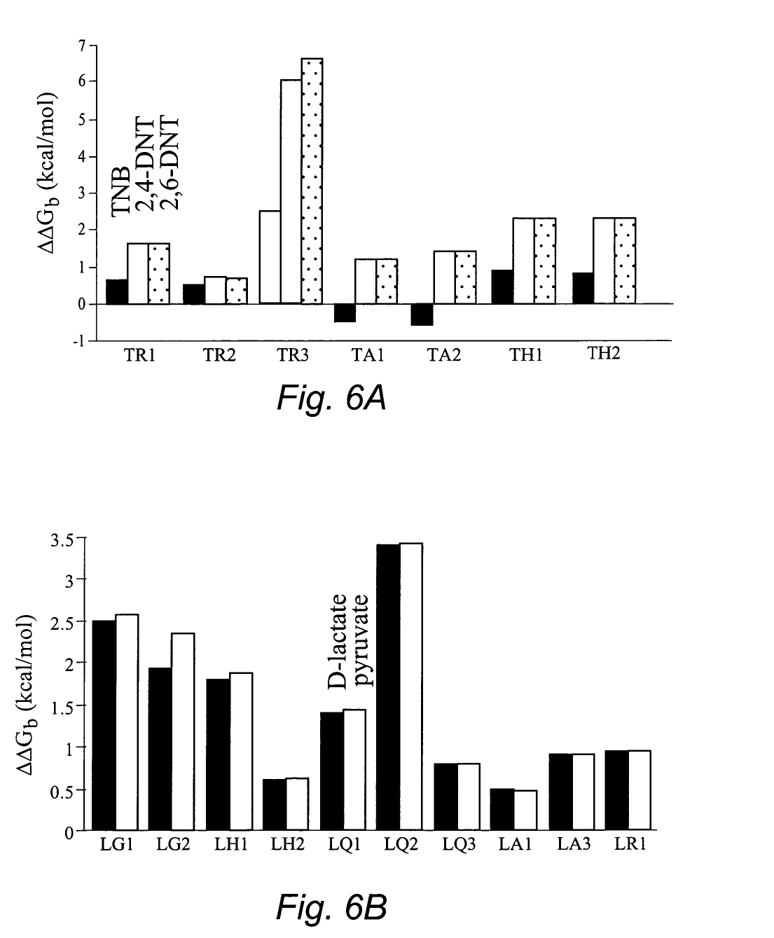

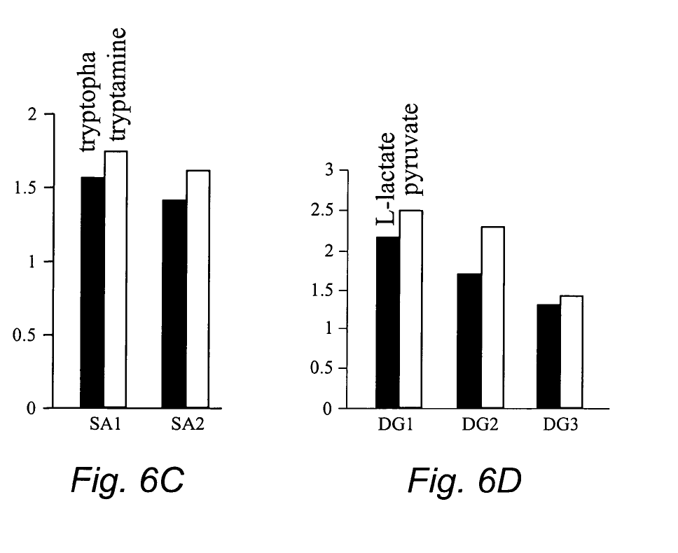

Every designed receptor exhibits detectable affinity for its target ligand. In the case of the TNT designs, all six receptors can distinguish the absence of a single nitro group (2,4- and 2,6-dinitrotoluene), and with the exception of the ABP design, the absence of a single methyl group (trinitrobenzene). Introduction of an additional point mutation suggested by visual inspection of the model to improve packing is sufficient to achieve the desired selectivity in this ABP design (FIG. 6). All ten L-lactate designs exhibit the desired chiral stereospecificity, selecting L-lactate over both the D-lactate enantiomer and pyruvate, the prochiral, oxidized form of lactate (FIG. 6). Similarly, all three D-lactate designs show specificity for D-lactate over L-lactate and pyruvate. The single serotonin design shows significantly lower affinity for tryptamine (absence of a hydroxyl) and tryptophan (absence of a hydroxyl, presence of a carboxylate). The relative free energy corresponding to the loss of a hydrogen bond in a decoy ligand (1-5 kcal/mol; FIG. 6) is consistent with the observed range of weak and strong hydrogen bonds (18). The automated computational design procedure therefore reliably predicts mutant receptors that attain ligand binding with the desired, drastically altered specificity, consistent with correct modelling of critical elements of molecular recognition: shape, functional groups, and chirality.

The affinities of the wild-type receptors for their cognate ligands fall in the 0.1 μM to 1.5 μM range (7). Two of the three TNT designs in RBP also fall into this range (Table 2); the binding behaviour of these computationally designed receptors is therefore indistinguishable from naturally evolved PBPs. It has been observed that the maximal binding affinity for many ligands is correlated with the number of non-hydrogen atoms (66). The affinity of one TNT design, TNT.R3, is 2 nM, corresponding to its empirically expected value. The single serotonin design does not attain the expected nanomolar affinity. The affinity of the fully automated design (Stn.A1) is 50 μM, and is improved to 4.7 μM by introduction of a single point mutation predicted to improve packing interactions between the receptor and ligand. Several of the lactate designs have micromolar affinities (one has slightly sub-micromolar affinity), approaching the expected maximal value for a six-atom ligand (0.3 μM).

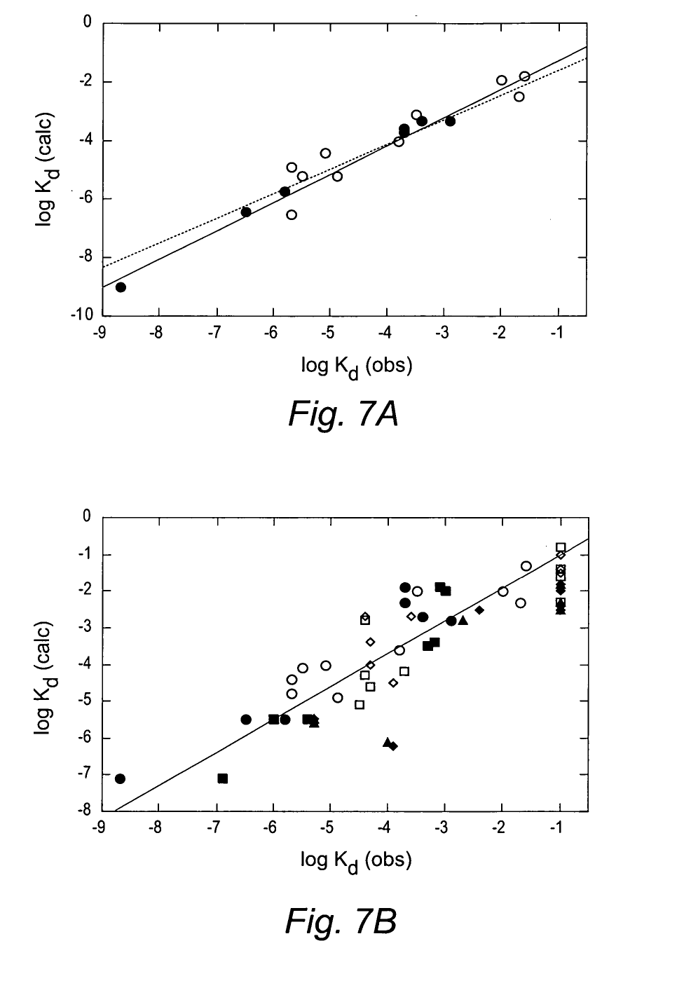

High-affinity receptors are successfully identified within the top ten ranked designs for each ligand, corresponding to a tiny fraction of the available search space. Nevertheless, the designs exhibit a significant spread in ligand-binding affinities, both for a given ligand in a particular scaffold, and between scaffolds. The likelihood that a protein scaffold can be mutated to accept a new target ligand (“adaptive potential”) is also variable. The observed range of affinities can be rationalized with an empirical quantitative structure-activity relationship (QSAR) that provides empirically fit weights for the DEE force-field components (steric clashes, unsatisfied hydrogen bonds) and takes into account additional factors not modelled by the DEE force field (hydrophobic contact areas, electrostatics, volume ratio of wild-type to target ligands as a measure of adaptive potential). This QSAR (FIG. 7) provides direct reciprocity between theory and experiment.

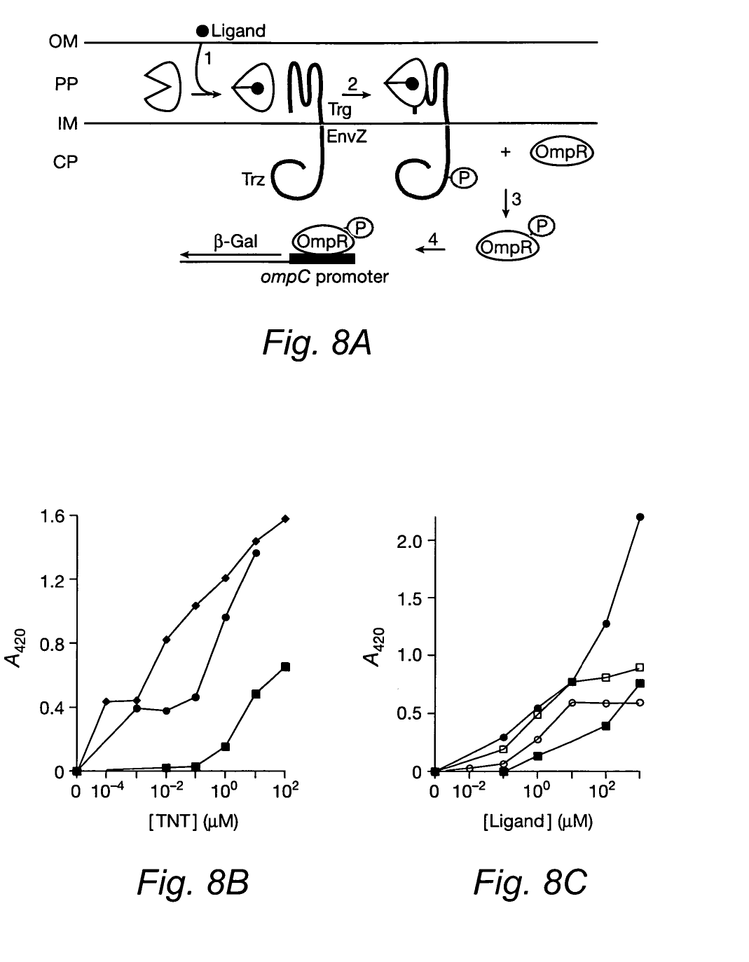

RBP and GBP control chemotaxis of E. coli towards sugars, mediated by a two-component signal transduction pathway (83). This response can be reconnected to gene regulation by constructing a synthetic signal transduction pathway that controls transcriptional upregulation of a β-galactosidase reporter gene (84) (FIG. 8A). The biological activities of the TNT and L-lactate designs in RBP and the L-lactate designs in GBP were tested in this pathway, replacing wild-type RBP and GBP with designed receptors. Wild-type receptors mediate increases in reporter gene expression in response to ribose or glucose, but not TNT or L-lactate. Conversely, all the redesigned receptors respond to their cognate, but not wild-type ligands. The dose-response curves of the TNT-binding RBP receptors follow the same order as the intrinsic ligand-binding affinities (FIG. 8B). The redesigned receptors therefore mediate signal transduction to extra-cellular TNT or L-lactate, as intended.

### EXAMPLE 2

Another application for the computational design of binding sites is the development of biosensors that detect chemical pollutants or threats. PMPA is a relatively nontoxic surrogate and the predominant hydrolytic degradation product of soman, a member of the organophosphate nerve agent family and a potent suicide inhibitor of acetylcholinesterase. It degrades rapidly upon exposure to water and forms PMPA. PMPA is only found following exposure to soman, and may even be present in the leading edge of a nerve agent cloud. Detection of PMPA is therefore important for weapons control, post-incident exposure determination and cleanup, and may prove useful as an attack indicator in a stand-off detector. Neither PMPA nor soman have an intrinsic chromophore or fluorophore. Therefore, a reagentless fluorescent biosensor for PMPA that responds rapidly and continuously is of great potential benefit for monitoring and control of this agent.

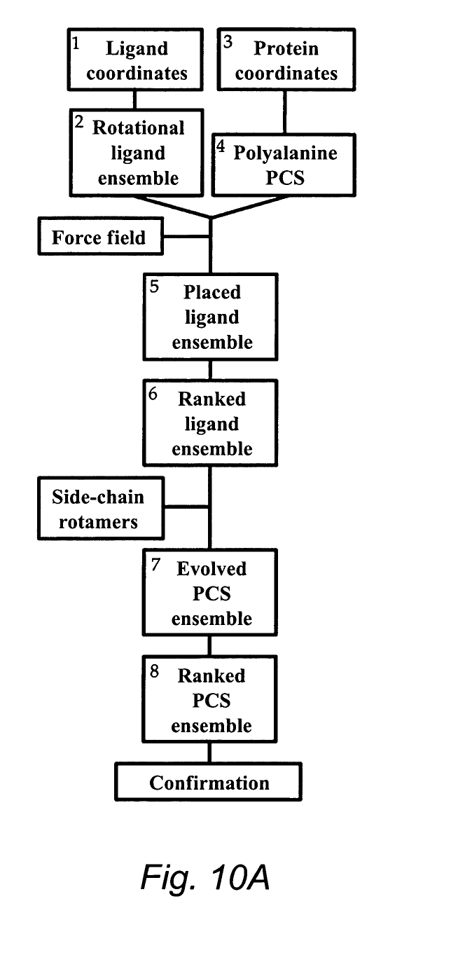

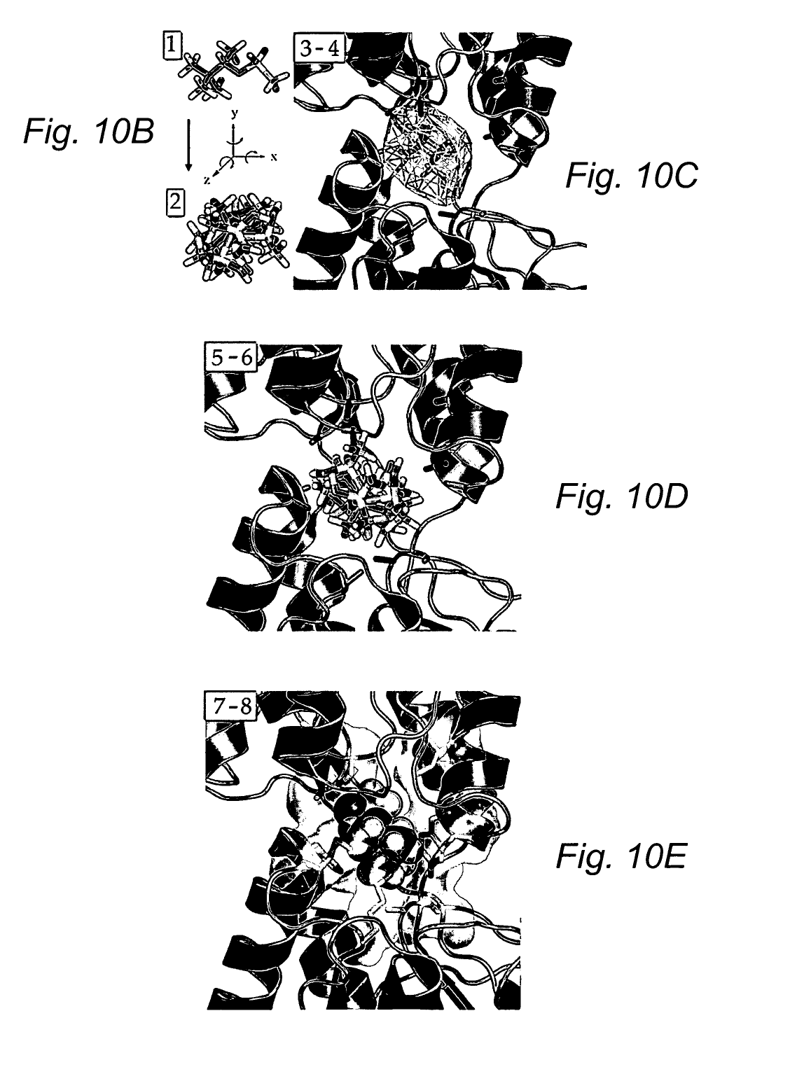

The ReceptorDesign component of the DEZYMER suite was used to generate designs of mutant receptors. This design process consisted of eight stages (FIG. 10). Stage 1: the internal degrees of freedom within the ligand are sampled to identify low-energy ligand conformations (the internal ligand ensemble, ILE). A single, minimum-energy conformer of the PMPA R-isomer was used in this study. Stage 2: a rotational ligand ensemble (RLE) is prepared in the absence of protein coordinates, sampling Eulerian rotations around the three principal molecular axes of the ligand (2.50 intervals, about 10⁶ poses). Stage 3: a pocket for the new binding site is identified, using the original ligand to locate the layer of residues that are in direct van der Waals or hydrogen bonding contact (the primary complementary surface, PCS). Stage 4: residues in the PCS (excepting glycines or prolines) are replaced with alanine, generating a truncated protein scaffold representing a PCS for which no sequence has been determined yet. Stage 5: the RLE is placed on each point of a cubic grid (0.5 Å spacing) within the convex hull which envelops the ligand van der Waals surface. Stage 6: a placed ligand ensemble (PLE) is constructed by selecting members from these RLEs that are sterically compatible with the truncated scaffold, and confined within the convex hull (>90% of ligand atoms). Stage 7: for each of top 10,000 docked ligands (selected from the PLE by choosing ligands with the fewest interactions with the truncated scaffold) a PCS is calculated. In this calculation, a side-chain rotamer library (an expanded version of (45) containing 6,122 rotamers) representing all possible mutations (except cysteine or proline) and side-chain conformations is placed at all positions in the PCS, and a sequence corresponding to the global minimum energy of a pairwise-decomposed potential function is identified by a dead-end elimination algorithm (24). This potential function is based on a semi-empirical force field that includes a modified Lennard-Jones potential to represent “fuzzy” van der Waals interactions (11, 24, 88) (parameters for amino acids and PMPA taken respectively from CHARM22 (43) or a universal force field approximation (42, 89)), an explicit geometry-dependent hydrogen-bonding term (11, 24, 88), a continuum solvation term to represent the hydrophobic effect with terms favoring or disfavoring burial of polar or nonpolar groups (11, 24, 88), and a linear term to account for differences in side-chain entropy (Eₛ=wRTInN, where N is the number of free torsions in the side chain, and w a weight; typically 1.0). Electrostatic contributions were not included in the calculations. The search algorithm maintains the ligand hydrogen bond inventory, selecting complementary sequences with minimal unsatisfied hydrogen bonds between ligand and protein. All PMPA oxygens were classified as hydrogen bond acceptors. Stage 8: the predicted designs were ranked by four independent criteria: van der Waals contacts, hydrogen bonding energies between protein and ligand, the number of unsatisfied ligand hydrogen bonds, and exposed cavities within the binding pocket. Suitable designs were selected by taking the intersection of the top 10% of each ranked list. This linear optimization method optimizes fitness functions with components of different magnitudes and ranges. The final choice is based on visual inspection of the molecular models. The design algorithm described here includes enhanced ligand sampling (stages 5 and 6) and introduction of the final selection by linear optimization (stage 8). The calculations were parallelized at stages 4 and 6, and carried out on a Beowulf cluster of twenty 1.7 GHz processors in about two days per combination of scaffold and ligand.

Mutations were introduced into the RBP and the GBP genes using overlap extension polymerase chain reaction (90). A single cysteine was introduced in each of the constructs (RBP: Cys 265; GBP: Cys 112) for covalent attachment of a fluorescent reporter (7). Constructs were cloned with a carboxy terminal decahistidine tag in a pET21 a expression vector using 5′ XbaI and 3′ EcoRI restriction sites. Mutations in the coding sequence were confirmed by DNA sequencing. Expression of mutant proteins was confirmed by MALDI-TOF mass spectrometry. His-tagged protein was purified by immobilized metal affinity chromatography on Ni⁺⁺ matrix and labeled with a reporter fluorophore conjugated through a thiol of the cysteine residue introduced near the hinge by site-directed mutagenesis. For GBP designs, all buffers contained 1 mM CaCl₂.

Ligand binding was measured by direct titration into a solution of covalently labeled protein (10 nM to 100 nM), and monitoring changes in fluorescence emission intensity at 25° C. (7).

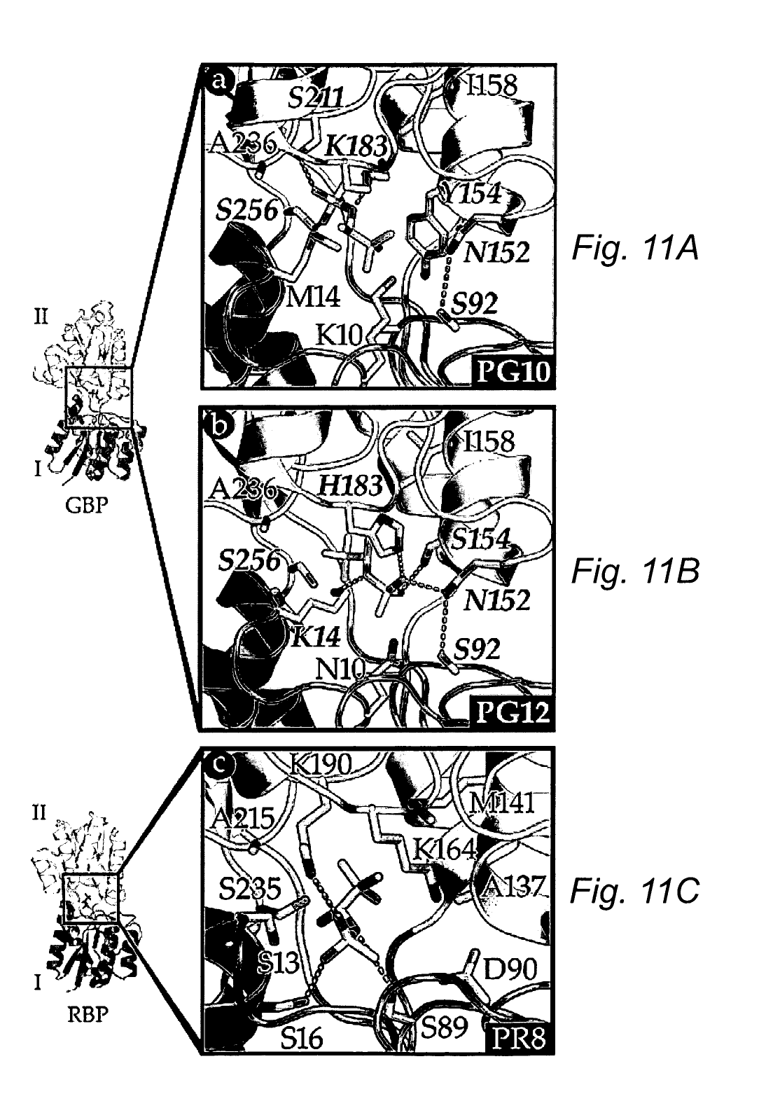

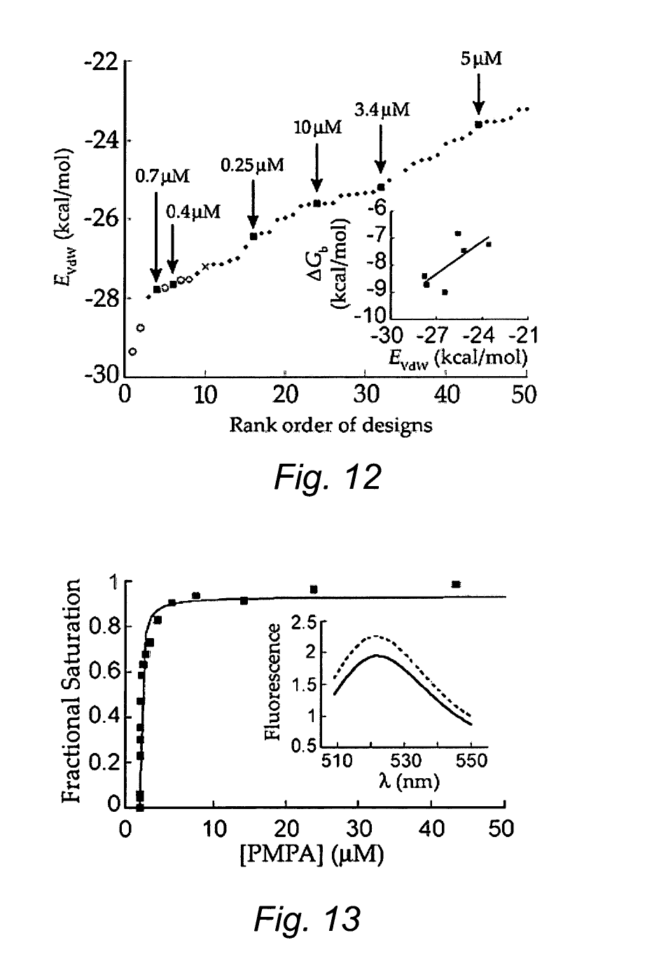

The binding pockets of RBP (PDB code: 2DRI) (91) and GBP (PDB code: 2GBP) (92) were redesigned to bind PMPA by the ReceptorDesign component of the DEZYMER suite, with eleven and twelve residues forming the primary complementary surface (PCS) in each receptor, respectively. The algorithm uses the three-dimensional structure of a protein to predict sequences and structures of binding sites that are complementary to a docked ligand (FIG. 10). A combinatorial search procedure simultaneously optimizes sequence choice and ligand docking to identify mutations that form complementary surfaces. Three RBP and twelve GBP designs were constructed by site-directed mutagenesis and their ligand-binding properties were determined (FIGS. 11-12; Table 3).

Each design corresponds to a separate PCS and a distinct orientation of the docked PMPA molecule. In all cases PMPA is sequestered within the binding site, with no direct contact with bulk solvent. In the majority of the designs the methyl phosphonate group points out towards the solvent. In the case of the PG10 design in GBP, however, this group is oriented inwards (FIG. 12). In all designs the hydrogen bonding potential of both phosphonate anionic oxygens as well as the phosphoester oxygen are satisfied.

The majority of the designs were built in GBP, and were selected from the top 50 ranked designs (FIG. 11), sampling both low- and high (er)-energy designs. The twelve PCS residues of the GBP designs can be divided into three groups according to the sequence diversity observed within the family of designs (Table 3): constant (92_{I}, 152_{II}, 236_{II}), highly conserved (211_{II}, 256_{II}), and variable (10_{I}, 14_{I}, 16_{I}, 91_{I}, 154_{II}, 158_{II}, 183_{II}). The constant and highly conserved positions all differ from the wild-type protein. Two of the three constant residues arise from a change in function between the designs and the wild-type receptor. In wild-type GBP Lys₉₂_{I}, and His152_{II} form hydrogen bonds to glucose. In most designed PMPA receptors Ser92_{I} and Asn152_{II} do not interact with the ligand (in PG12 Asn152_{II} forms an additional hydrogen bond with PMPA), but participate in a hydrogen-bonding network connecting the N— and C-terminal domains. This network may function as a “latch” that stabilizes the closed form (FIGS. 11A-B). The third constant residue (Ala236_{II}) is constrained by steric differences between glucose and PMPA. In wild-type GBP, Asp236_{II} forms a hydrogen bond to glucose; in all designs the PMPA position precludes choice of any amino acid but alanine or glycine at this position. The highly conserved positions 21_{II} (Ser or Asn) and 256_{II} (Ser or His) also have switched from ligand binding (Asn211_{III} and Asn256_{II} interact with the O3 and O4 glucose hydroxyls respectively) to structural functions. In eleven designs Ser211_{II} forms a hydrogen bond with the main-chain carbonyl of position Val235_{II}; in three designs Asn211_{II} interacts both with the amide protein of Met214₁l and the carbonyl of His183_{II}. In the majority of the designs Ser256_{II} forms a hydrogen bond with Gln261_{II} outside the PCS (with the exception of PG10, where Ser211_{II} forms a hydrogen bond to PMPA).

The designs leave a cavity between Ser256_{II} and the PMPA pinacolyl group. The penalty for solvent accessibility of the hydrophobic ligand moiety apparently was insufficient to overcome the reward for forming the inter-residue hydrogen bond. We constructed additional point mutations at position 256_{II} in designs PG4 and PG12 to fill this cavity (PG4_{—}256F and PG12_{—}256F; Table 3).

Sequences at the variable positions are diverse: on average 33% of the residues differ among the designs, reflecting alternative ways for providing hydrogen bonds and hydrophobic surfaces. The designs vary in their PCS positions at which hydrogen-bonding side chains are placed.

The three designs constructed in RBP also exhibit variations in sequence diversity and residue function switching. In PR8 Ser235_{II} is associated with a defect analogous to Ser256_{II} in GBP. Ser235_{II} makes no direct contacts with PMPA, but forms a hydrogen bond with the hydroxyl of Ser103_{II}, resulting in a cavity near the pinacolyl group. To fill this cavity, additional point mutations were constructed in the RBP design PR8 at position 235_{II} (Table 3).

All three RBP designs and ten of the twelve primary GBP designs expressed soluble protein; one GBP design did not express, while another precipitated upon purification. Several of the mutants were less stable than the parent proteins (GBP, 58° C.; RBP, 60° C.), having thermo-stabilities that range between 32° C. to 58° C. as determined by thermal denaturation, monitoring circular dichroism (88).

Of the eighteen fluorescent conjugates prepared by labeling with thiol-reactive fluorophores at Cys256 (RBP) or Cys112 (GBP), twelve show changes in fluorescence upon addition of PMPA (FIG. 13). Neither wild-type RBP nor GBP conjugates respond to PMPA.

Observed PMPA affinities range from 68 nM (PR8) to 10 μM (PG18) (Table 3). Some of the cavity-filling mutations constructed at position 235_{II} in RBP show improvements in affinity. Phenylalanine at position 235_{II} increases the affinity of the receptor for PMPA (K_{d}=45 nM), while Ala235_{II}, or Ile235_{II} have no effect (Table 3). The equivalent mutation at position 256_{II} in GBP (PG4_{—}256F, K_{d}=0.4 μM; PG12_{—}256F, K_{d}=0.11 μM) has similar effects on binding.

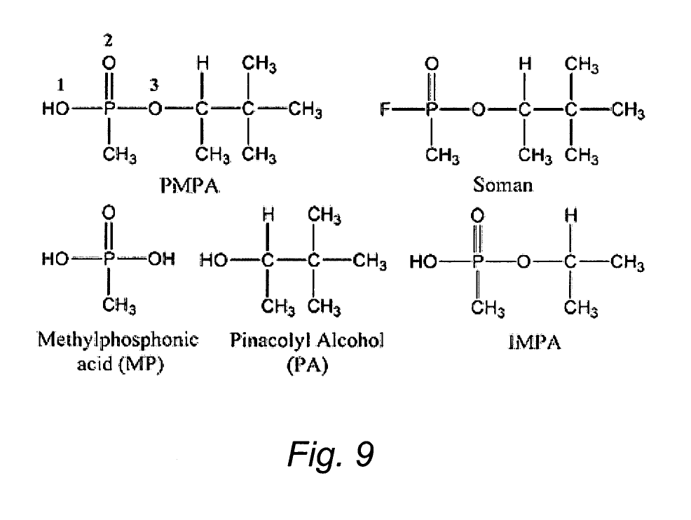

The ligand-binding specificity of two designs was tested by measuring affinities for isopropyl methyl phosphonic acid (IMPA) (FIG. 9), the hydrolysis product of the nerve agent sarin. PG10 and PG12 bind IMPA approximately 10-fold less tightly that PMPA (K_{d}=7 μM and K_{d}=2 μM respectively), indicating significant discrimination between the aliphatic groups of the two molecules.

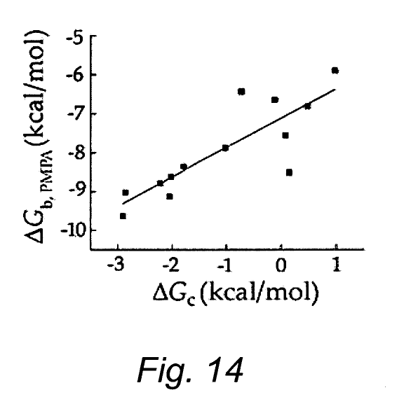

The affinities of the designs for pinacolyl alcohol (PA) and methyl phosphonate (MP), representing the aliphatic and hydrophilic moieties of PMPA respectively (FIG. 9), were determined (Table 3). The K_{d} values of the receptors for PA and MP are 10²-10⁴ and 10⁴-10⁵-fold higher than those for PMPA, respectively. A coupling energy (93), ΔG_{c}, can be defined as: ΔG_{c}=ΔG_{b,pMPA}−(ΔG_{b.PA}+ΔG_{b.MP}), where ΔG_{b.PMPA}, ΔG_{b,PA}, ΔG_{b.MP} are the binding energies (RT In K_{d}) for PMPA, PA, and MP, respectively. Favorable inter-fragment interactions result in ΔG_{c}<0, unfavorable ΔG_{c}>0. Analysis of fragment binding is typically used to assess strain or entropic factors within a ligand (93). Here ΔG_{c} values between designs are interpreted as strain within the designed proteins, reflecting differences in the structural complementarity between a to design and its bound ligand. FIG. 14 reveals a positive correlation between ΔG_{c} and the affinity for PMPA: as ΔG_{c} decreases, ΔG_{b,PMPA} becomes more favorable. Decreases in fragment strain therefore correlate with increased receptor affinities, and indicate differences in the complementtary of the designed surfaces.

The contributions of specific interactions were tested by alanine scanning mutagenesis in two designs, PG10 and PG12 (Table 4) (94), which bind PMPA in opposite orientations. In the PG10 design (MP moiety points inwards) mutation of predicted hydrogen bonds to an anionic oxygen (O1, PG10_S211A) or the phosphoester oxygen (O3, PG10_K183A) results in a 2.1 and 2.4 kcal/mol loss of binding energy respectively, consistent with typical hydrogen bonding contributions (18). Loss of the predicted interaction between Ser256_{II} and the other anionic oxygen has no appreciable effect (O2, PG10_S256A), potentially indicating that this hydrogen bond is absent. Ser256_{II} is also predicted to form a hydrogen bond with Gln261_{II}. The two interactions therefore may compete rather than co-exist. In the PG12 design (MP moiety points outwards), loss of mutation of predicted hydrogen bonds to the anionic oxygens (O1, PG12N152A; O2, PG12_S154A; O1 PG12_{—H}183A) results in a 2-3 kcal/mol loss of binding energy, consistent with the model (Table 4).

Van der Waals interactions were also investigated. In PG10 Tyr154_{II} interacts with the pinacolyl moiety of PMPA and hydrogen bonds to Thr110_{II}. Loss of these predicted interactions decreases binding by 2.4 kcal/mol (Table 4). Furthermore, binding of PA, but not MP is affected consistent with the orientation of PMPA in the model. Similarly, in PG12, Asn211_{II} forms van der Waals interactions with the pinacolyl moiety and hydrogen bonds to the backbone carbonyl of position 214_{II}. Loss of these predicted interactions (PG12_N211A) results in a decreased affinity for PMPA, but to a lesser extent (0.9 kcal/mol) than is observed for the Tyr154_{III} in PG10. Again, as expected, PA, but not MP binding is affected.

Alanine-scanning mutagenesis has also demonstrated that the inter-domain latch, contributed by constant residues Ser92_{I} and Asn152_{II}, is important for binding (Table 4). Mutations of either residue decrease binding, as expected for the removal of an interaction that stabilizes the closed state (95, 96).

The Ser256_{II}Ala mutation in PG12 exhibits the largest change in affinity (4 kcal/mol) (Table 4). This residue is not predicted to interact directly with PMPA, instead it hydrogen bonds to Gln261_{II} leaving a cavity. Enlargement of this putative cavity in the alanine mutation is predicted to trap water near the hydrophobic pinacolyl moiety, thereby decreasing the affinity for PMPA. Loss of PA and retention of MP binding in this mutant is observed and is consistent with this interpretation.

The designs introduced 9 to 12 mutations in the parent proteins. Twelve of twenty designs tested exhibited PMPA-dependent changes in emission intensity of a fluorescent reporter with affinities between 45 nM and 10 μM. The contributions to ligand binding by individual residues were determined in two designs by alanine-scanning mutagenesis, and are consistent with the molecular models. These results demonstrate that designed receptors with radically altered binding specificities and affinities that rival or exceed those of the parent proteins can be successfully predicted. The designs vary in parent scaffold, sequence diversity, and orientation of docked ligand, suggesting that the number of possible solutions to the design problem is large and degenerate. This observation has implications for the genesis of biological function by random mutagenic processes.

About 50% of the computer-generated designs show PMPA-mediated changes in fluorescence of the covalently coupled reporter groups (57% if designs that do not express or that precipitate are discounted). This success rate represents a lower bound, because false negatives can arise if the equilibrium between the open and closed states is sufficiently altered to preclude their interconversion, or if the fluorophore no longer interacts differentially with these two conformations.

PMPA affinities of the designed receptors range from 45 nM to 10 μM. RBP and GBP bind their cognate sugars with 0.2 μM and 0.5 μM affinities respectively (7). Empirical limits have been established for the ligand affinities of naturally evolved proteins (97). For PMPA this limit ranges from about 2 nM to about 1 μM. The affinities of many designs reported here fall within this range and rival or exceed those of the parent receptors.

Selected designs sample both high- and lower-ranked candidates. Designs selected from the top 20 exhibit higher affinities for PMPA than those selected from lower-ranked designs (FIG. 12). Analysis of the affinities for PA and MP suggests that the designed receptors differ in the strain they impose upon the ligand (FIG. 14) (93).

The effects of individual alanine mutations on PMPA binding in designs PG10 and PG12 are mostly consistent with the predicted interactions. Furthermore, the designed receptors distinguish steric differences between the aliphatic moieties of PMPA and IMPA (FIG. 9). We therefore conclude that predicted molecular models of the designs are largely correct.

The designs contain defects, indicating that the computational design methods require further improvements. Virtually all designs have a cavity between the protein and bound ligand in the vicinity of the hinge region. This cavity defect is likely to be a consequence of inaccurate modeling of relative contributions by hydrogen bonds, polar group burial, solvent accessibility, and omission of electrostatic contributions. Nevertheless, the experimentally validated ligand-binding properties of the designs reported here demonstrate that even relatively simple representations of atomic interactions are sufficiently powerful to capture dominant effects of biomolecular recognition in design calculations.

The designed PCS has fewer residues that make direct contacts with the ligand than those in the wild-type receptors. Consequently, a significant fraction of the side chains switch function from ligand binding in the wild-type receptor to a structural role in the designed receptors and lack sequence diversity. The residues that interact directly with the ligand, however, are highly diverse and depend on the orientation of the bound ligand. Thus even in this small set of designs, significant diversity in structure and sequence is observed, suggesting that solutions to the design problem are highly degenerate. These observations presumably reflect a fundamental characteristic of protein sequences, since potential diversity is an essential prerequisite for the genesis of function by the random processes of organic evolution (98).

The receptors described here can function as reagentless fluorescent biosensors for PMPA with a lower detection limit of about 4 nM (about 1 ppb). Given the structural similarities between soman and PMPA, the designed receptors are likely to bind soman with affinities similar to those of PMPA. The detection limit is probably sufficient for the development of stand-off or post-incident detectors of soman, and rivals the lower limits of current methods. Unlike acetylcholinesterase-based assays, the designed receptors described here do not rely on the presence of soman, which rapidly degrades to form PMPA. Other techniques require several components and longer preparation, incubation, and detection times. A reagentless fluorescence biosensor has significant advantages such as rapidity of the fluorescent response, reversibility, and simplicity. The molecular recognition element in a deployable biosensor must be sufficiently robust to withstand field conditions. The designed receptors reported here do not yet meet this standard, since their thermostability may not be sufficiently high. Nevertheless, computationally designed receptors represent an initial stage in the development of a novel class of biosensors for the rapid, continuous, and accurate detection of nerve agents.

### EXAMPLE 3

Enzymes are amongst the most proficient catalysts known (99), and catalyze a wide variety of reactions in aqueous solutions under ambient conditions with exquisite selectivity and stereospecificity. Catalysis takes place in tailored pockets that simultaneously optimize binding of reactants, intermediates, transition states, and products, orient reactive residues, stabilize transition states, select catalytically competent substrate conformations, and dynamically interconvert between microstates (100, 101). The rational design of enzymes has tremendous practical potential for developing novel synthetic routes (73, 102), but presents a formidable challenge and is one of the most stringent tests for understanding protein chemistry. Here we present structure-based computational design techniques that predict mutations for the construction of catalytically active sites in proteins of known structure. Using these methods, we converted ribose-binding protein (62) into analogs (NovoTims) of the glycolytic enzyme triose phosphate isomerase (103). Several NovoTims exhibit rate enhancements of approximately 10⁵ to 10⁶ and are biologically active, supporting growth of Escherichia coli under gluconeogenic conditions. The inherent generality of computational design implies that it may be possible to design many enzymes by this approach.

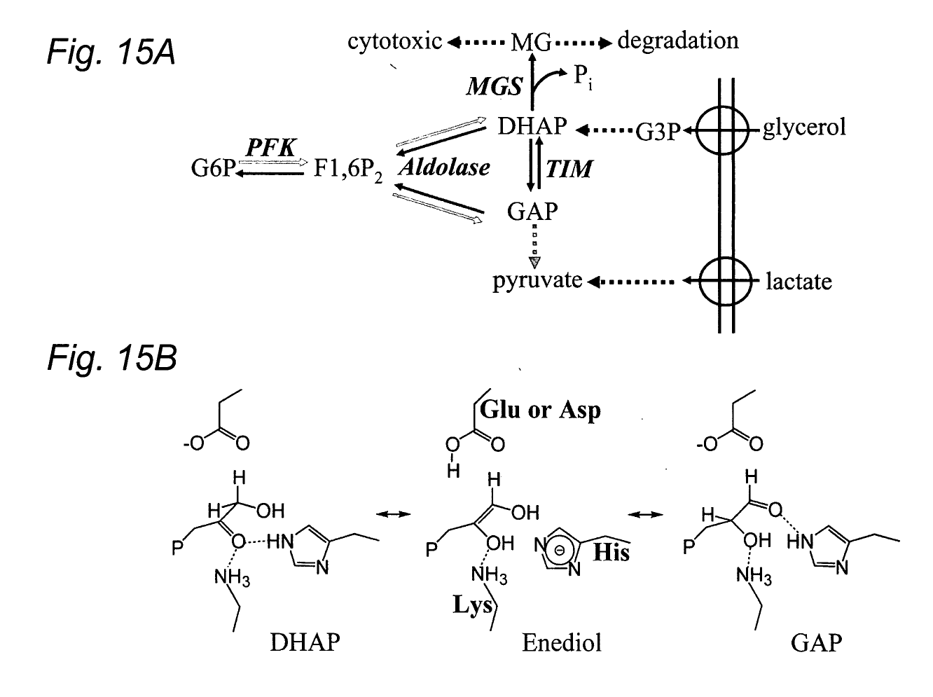

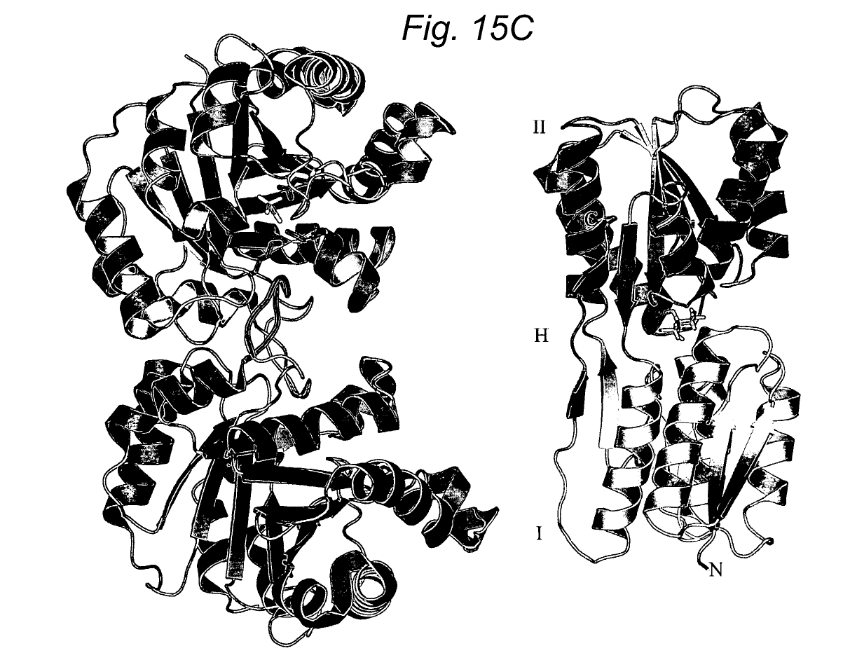

Triose phosphate isomerase (TIM) is an essential component of the Embden-Meyerhof pathway (104), interconverting dihydroxyacetone phosphate (DHAP) and glyceraldehyde-3-phospate (GAP) (FIG. 15A). In glycolysis TIM channels these two triose phosphate products of aldolase into pyruvate; in gluconeogenesis TIM ensures that both substrates are supplied to aldolase. The isomerization reaction involves two successive proton exchanges (103) (FIG. 15B), and is considered an archetype for proton transfer chemistry, which is central to many enzyme mechanisms (105). Extensive studies support a mechanism (103) whereby a carboxylate abstracts the DHAP pro-R proton at C1 to form a cis-enediol(ate) intermediate, followed by imidazole-mediated proton transfer between the C1 and C2 oxygens, yielding GAP. The C1 proton pKₐ of about 18 imposes a large barrier to proton abstraction (106), which is overcome by a low-barrier hydrogen bond (107) (LBHB) that requires precise functional group alignment (108-110). Transition states are further stabilized electrostatically by lysine (109, 110). TIM also selects a substrate conformation that minimizes alignment of the enediolate double bond and phosphate 7t systems, thereby stereoelectronically disfavoring an undesirable β-elimination of the phosphate (111) that produces methylglyoxal (MG) which is cytotoxic in excess (112). A mobile loop permits substrate access and sequesters the reaction from solvent (113) (FIG. 15C). The TIM reaction therefore presents a complex design target demanding simultaneous capture of many mechanistic principles: acid-base catalysis, transition state stabilization, reactive group alignment, low-barrier hydrogen bonds, stereoelectronic control by ground state selection, electrostatic effects, and protein dynamics.

Here we demonstrate that structure-based computational design techniques can be used to introduce isomerase activity into the bacterial ribose-binding protein (RBP) which is a periplasmic receptor that has no known catalytic activity. RBP is a monomer and consists of two domains linked by a hinge region (62) (FIG. 15C). The protein adopts two conformations, a ligand-free open form and a ligand-bound closed form, which interconvert via hinge-bending motions. Analogous to TIM, the ribose ligand is sequestered from solvent in the closed form. TIM is a homodimer of α/β barrel monomers (109, 110) (FIG. 15C). RBP and TIM structures fall into different topological classes. Introduction of TIM activity into RBP is therefore equivalent to convergent evolution by computational design.

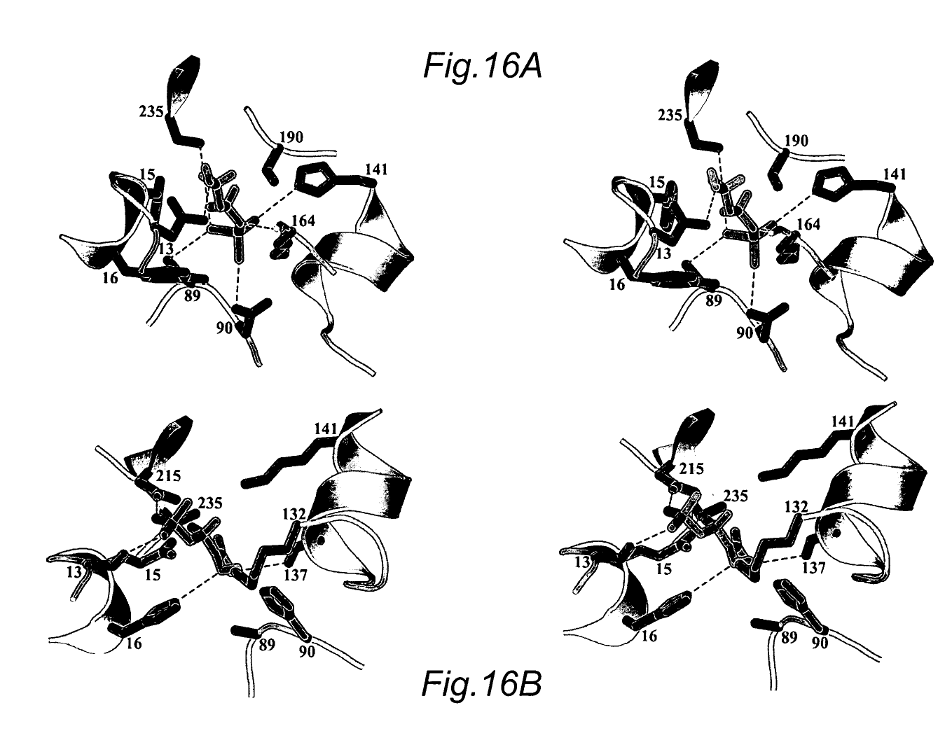

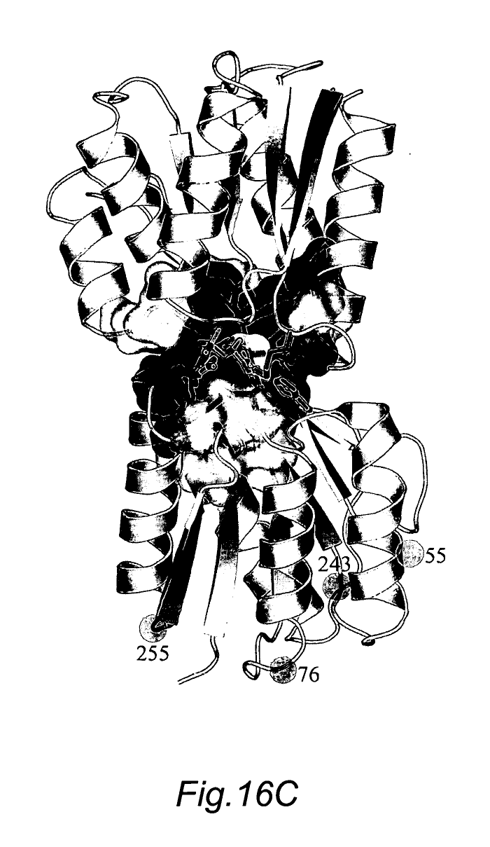

Initially we tested whether RBP can be redesigned to bind GAP and DHAP, without regard to catalytic activity. The design algorithm predicted mutations that convert RBP (PDB code: 2DRI for wild-type sequence) into a receptor for DHAP by changing the layer of residues directly contacting ribose in the wild-type protein structure. Sequences that form stereochemically complementary ligand-binding surfaces were identified using a combinatorial optimization algorithm that integrates ligand docking and placement of amino acid side-chain rotamer libraries to locate energetic minima in a potential function incorporating van der Waals, hydrogen bonding, solvation, and electrostatic interactions (87) between the amino acids and ligand. Four designs bind DHAP and GAP with micromolar affinities (FIG. 16A) but exhibit no TIM activity. This experiment shows that RBP can be mutated to bind both substrates, which is a necessary preliminary finding prior to the introduction of catalysis.

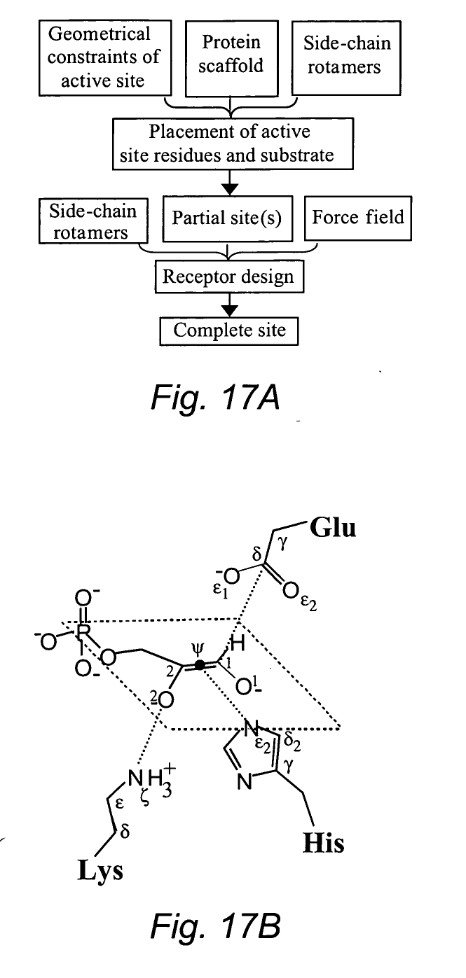

To include catalytic activity in the receptor, we developed a new procedure that introduces catalytically active residues into the receptor design process (FIG. 17A). First, a geometrical definition of key interactions contributing to catalysis is generated. Second, a combinatorial search algorithm (85) identifies positions where placement of catalytic residues and substrate simultaneously satisfies these geometrical constraints. Third, the remainder of the complementary surface is generated around the placed substrate using the receptor design algorithm. Designs were generated using the allowed geometrical relationships between the enediolate reaction intermediate, glutamate, histidine, and lysine as a minimalist model of interactions that are critical to catalysis (FIG. 17B). We tested fourteen designs subdivided into three families that differ in placement of these three catalytic residues (Table 5). Seven designs show increases in GAP production over background. One design, NovoTim1.0, is significantly more active. It exhibits saturation kinetics (Table 6), and is competitively inhibited by phosphoglycolate (Kᵢ=130 μM), a known inhibitor of wild-type TIM (103) (Kᵢ=4 μM).

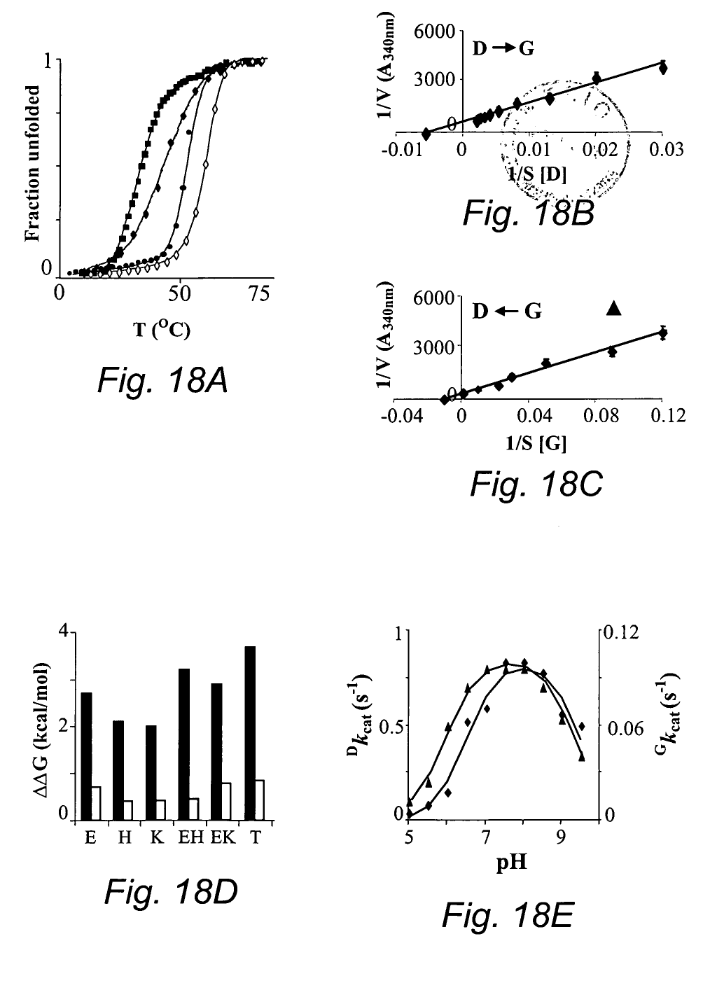

NovoTim1.0 is less thermostable than the parent protein (FIG. 18A). We postulated that steric imperfections in the interactions between the designed binding surface residues and the surrounding protein matrix cause this decreased stability. Previously, we have established that in RBP-based metalloprotein designs, stability is restored by designing mutations in residue layers surrounding designed binding surfaces (114). We redesigned NovoTim1.0 in a similar manner (FIG. 18C; Table 5). In NovoTim1.1, the thirteen original mutations were retained and nine additional ones identified by computational design, which increased the stability by 5° C. In NovoTim1.2, only the three catalytic residues were retained, and the sequences of the nine binding and nine interfacial residues were (re-)designed together. NovoTim1.2 stability is increased by 15° C., approaching that of the parent protein. NovoTim1.1 has similar kinetic properties as NovoTim1.0, whereas in NovoTim1.2 k_{cat} and K_{M} each has improved approximately two-fold (FIG. 18B; Table 6).

At least 95% of DHAP (GAP) is converted into GAP (DHAP) in the reaction catalyzed by NovoTims, as judged by NADH (NAD⁺) production. The loss of enzyme activity observed in single, double, and triple alanine mutants of NovoTim1.2 indicates that all three designed catalytic residues make critical contributions to catalysis (FIG. 18C). The pH dependencies of the forward and reverse reactions catalyzed by NovoTim1.2 are similar to wild-type TIM (FIG. 18D). These results show that the desired reaction is predominant, the designed catalytic groups are key to the enzyme mechanism, and the active site microenvironment approximates the naturally evolved enzyme.

In E. coli, gluconeogenic growth on lactate or glycerol requires TIM activity (FIG. 15A). Glycerol feeds into DHAP and places more stringent demands than lactate on TIM activity, because elevated DHAP levels increase cytotoxic MG production, which is mitigated through TIM-mediated conversion of DHAP into GAP (112). Complementation of a TIM-deficient strain (104), DF502, by over-expressed NovoTims was tested on both gluconeogenic substrates (115) in the presence and absence of the inducer isopropyl-β-D-thiogalactopyranoside (IPTG). NovoTims1.0 and 1.2 (1.1 not tested) support IPTG-dependent growth on lactate, but not glycerol. NovoTim1.2 was further mutagenized by an error-prone polymerase chain reaction (116), and mutants were selected on glycerol. Four isolates were obtained from approximately 10⁵ transformants. The different mutations in NovoTims1.2.1-1.2.4 are localized on the protein surface (FIG. 16C) and improve k_{cat} and K_{M} values, with the largest changes corresponding to two-fold and three-fold increases in k_{cat} and k_{cat}/K_{M} values respectively.

We have successfully converted a protein devoid of catalytic activity into a triose phosphate isomerase, using computational design techniques to predict 13 to 21 mutations that introduce three catalytically active residues together with a stereochemically complementary substrate-binding surface. This minimalist design is based on key short-range interactions observed in naturally evolved TIMs, and is sufficient to increase the NovoTim-catalyzed reaction 10⁵-fold to 10⁶-fold over background. This rate enhancement is the largest reported for rationally designed enzymes (73, 102). NovoTim1.2 is sufficiently active to support growth under permissive gluconeogenic conditions, and requires only small improvements to support full biological activity. Nevertheless, the k_{cat} and k_{cat}/K_{M} values of NovoTim1.2.1 are 2,700-fold and 220-fold less than wild-type TIM, whose apparent second-order rate constant approaches the diffusion-limited encounter of enzyme with substrate (103). Alanine-scanning mutagenesis indicates that all residues designed to be catalytically active contribute significantly to rate enhancement. Furthermore, the electrostatic microenvironment as probed by pH dependence of k_{cat} is similar to the wild-type enzyme. However, it is likely that NovoTims have a sub-optimal hydrogen bond between the catalytic glutamate and substrate C1 proton, which is a critical feature of the TIM reaction mechanism (108-110) (we note that shortening of glutamate to aspartate in the wild-type enzyme (117), presumably destroying the LBHB, results in a mutant with similar activity as NovoTims). Elaboration of the minimalist mechanism in future designs will allow testing of other contributions to rate enhancement, such as protein dynamics and long-range electrostatics.

Rational design of enzymes is a stringent test of our understanding of protein chemistry and has numerous potential applications. Here we present and experimentally validate the computational design of enzyme activity in proteins of known structure. We have predicted mutations that introduce triose phosphate isomerase activity into ribose-binding protein, a receptor that is normally devoid of enzyme activity. The resulting designs contain 18 to 22 mutations, exhibit 10⁵-fold to 10⁶-fold rate enhancements over the uncatalyzed reaction, and are biologically active, supporting growth of Escherichia coli under gluconeogenic conditions.

The combined placement of mechanistically critical residues with construction of a surface that is stereochemically complementary to the entire substrate (and product) is a critical aspect of the design method presented here. This capability was absent in previously reported attempts at enzyme design (73) and is likely to be the main reason for the much higher rate enhancements and apparent second order rate constants observed in this study. With the predicttion accuracies now within reach of computational protein design (11, 118), and introduction of increasing levels of mechanistic detail and sophistication in future designs, this design process can be extended to other substrates and reactions using our knowledge of catalytically active residues and well-known principles of enzyme chemistry (122).

### References

1. Bishop et al. (2000) Annu. Rev. Biophys. Biomol. Struct. 29, 577-606.

2. Harris & Craik (1998) Curr. Opin. Chem. Biol. 2, 127-132.

3. Bolon & Mayo (2001) Proc. Natl. Acad. Sci. USA 98, 14274-14279.

4. Benson et al. (2000) Proc. Natl. Acad. Sci. USA 97, 6292-6297.

5. Benson et al. (2002) Biochemistry 41, 3262-3267.

6. Hellinga & Marvin (1998) Trends Biotechnol. 16, 183-189.

7. de Lorimier et al. (2002) Protein Sci. 11, 2655-2675.

8. Hasty et al. (2002) Nature 420, 224-230.

9. Koh (2002) Chem. Biol. 9, 17-23.

10. Maier et al. (2001) J. Chromatogr. A 906, 3-33.

11. Looger et al. (2003) Nature 423, 185-190.

12. Marazuela & Moreno-Bondi (2002) Anal. Bioanal. Chem. 372, 664-682.

13. Kipriyanov & Little (1999) Mol. Biotechnol. 12, 173-201.

14. Arnold (2001) Nature 409, 253-257.

15. Olsen et al. (2000) Nature Biotechnol. 18, 1071-1074.

16. Dahiyat & Mayo (1997) Science 278, 82-87.

17. DeGrado et al. (1999) Annu. Rev. Biochem. 68, 779-819.

18. Fersht (1999) Structure and Mechanism in Protein Science (Freeman, New York).

19. Sundberg et al. (2000) Biochemistry 39, 15375-15387.

20. Sinha & Smith-Gill (2002) Curr. Prot. Pept. Sci. 3, 601-614.

21. Slagle et al. (1994) J. Biomol. Struct. Dyn. 12, 439-456.

22. Babine & Bender (1997) Chem. Rev. 97, 1359-1472.

23. Gordon et al. (1999) Curr. Opin. Struct. Biol. 9, 509-513.

24. Looger & Hellinga (2001) J. Mol. Biol. 307, 429-445.

25. Reina et al. (2002) Nature Struct. Biol. 9, 621-627.

26. Berman et al. (2000) Nucleic Acids Res. 28, 235-242.

27. EU 3-D Validation Network (1998) J. Mol. Biol. 276,417-436.

28. Laskowski et al. (1996) J. Biomol. NMR 8, 477-486.

29. Kini & Evans (1991) J. Biomol. Struct. Dyn. 9, 475-488.

30. Tann et al. (2001) Curr. Opin. Chem. Biol. 5, 696-704.

31. Scott & Tanaka (1998) Methods Enzymol. 293, 620-647.

32. Osguthorpe (2000) Curr. Opin. Struct. Biol. 10, 146-152.

33. Huang et al. (1996) Biochemistry 35, 3439-3446.

34. Marvin & Hellinga (2001) Proc. Natl. Acad. Sci. USA 98,4955-4960.

35. Allen (2002) Acta Crystallogr. B 58, 380-388.

36. Van Drie et al. (1989) J. Comput. Aided Mol. Des. 3, 225-251.

37. Yoshida et al. (2003) J. Comput. Chem. 24, 319-327.

38. Stewart (1990) J. Comput. Aided Mol. Des. 4, 1-105.

39. Shrake & Rupley (1973) J. Mol. Biol. 79, 351-371.

40. Pearlman & Kim (1985) Biopolymers 24, 327-357.

41. Mills & Dean (1996) J. Comput. Aided Mol. Des. 10, 607-622.

42. Rappe et al. (1992) J. Am. Chem. Soc. 114, 10024-10035.

43. MacKerell et al. (1998) J. Phys. Chem. B 102, 3586-3616.

44. Dunbrack (2002) Curr. Opin. Struct. Biol. 12, 431-440.

45. Lovell et al. (2000) Proteins 40, 389-408.

46. Zhang (1999) Proteins 34, 464-471.

47. Ying & Kim (2002) J. Biomech. 35,1647-1657.

48. Fritzer (2001) Spectrochim. Acta A 57, 1919-1930.

49. Badel-Chagnon et al. (1994) J. Mol. Graph. 12, 162-168, 193.

50. Jiang et al. (2000) Protein Sci. 9, 403-416.

51. Kuhlman et al. (2002) J. Mol. Biol. 315, 471-477.

52. Desjarlais & Handel (1999) J. Mol. Biol. 290, 305-318.

53. Offredi et al. (2003) J. Mol. Biol. 325, 163-174.

54. Desmet et al. (2002) Proteins 48, 31-43.

55. Ulmer (1983) Science 219, 666-671.

56. Jencks (1975) Adv. Enzymol. 43, 219-240.

57. Fischer et al. (1995) J. Mol. Biol. 248, 459-477.

58. Srivastava & Crippen (1993) J. Med. Chem. 36, 3572-3579.

59. Desmet et al. (1992) Nature 356, 539-542.

60. Tam & Saier (1993) Microbiol. Rev. 57, 320-346.

61. Vyas et al. (1994) Biochemistry 33, 4762-4768.

62. Mowbray & Cole (1992) J. Mol. Biol. 225, 155-175.

63. Quiocho & Vyas (1984) Nature 310, 381-386.

64. Sun et al. (1998) J. Mol. Biol. 278, 219-229.

65. Yao et al. (1994) Biochemistry 33, 4769-4779.

66. Kuntz et al. (1999) Proc. Natl. Acad. Sci. USA 96, 9997-10002.

67. Mehrvar et al. (2000) Anal. Sci. 16, 677-692.

68. Daunert et al. (2000) Chem. Rev. 100, 2705-2738.

69. Ward (2000) Anal. Chem. 72, 4521-4528.

70. Alaimo et al. (2001) Curr. Opin. Chem. Biol. 5, 360-367.

71. Clackson (2000) Gene Ther. 7, 120-125.

72. Doyle et al. (2000) Curr. Opin. Struct. Biol. 4, 60-63.

73. Bolon et al. (2002) Curr. Opin. Chem. Biol. 6, 125-129.

74. Tam & Saier (1993) Res. Microbiol. 144, 165-169.

75. Aranda & Pascual (2001) Physiol. Rev. 81, 1269-1304.

76. Kirkham et al. (1999) J. Mol. Biol. 285, 909-915.

77. Smith et al. (2002) J. Mol. Biol. 319, 807-821.

78. Hertzel & Bernlohr (2000) Trends Endocrinol. Metab. 11, 175-180.

79. Marvin et al. (1997) Proc. Natl. Acad. Sci. USA 94, 4366-4371.

80. Marvin & Hellinga (1998) J. Am. Chem. Soc. 120, 7-11.

81. Benson et al. (2001) Science 293, 1641-1644.

82. U.S. Food & Drug Administration. (1992) Chirality 4, 338-340.

83. Stock et al. (2000) Annu. Rev. Biochem. 69, 183-215.

84. Baumgartner et al. (1994) J. Bacteriol. 176, 1157-1163.

85. Hellinga & Richards (1991) J. Mol. Biol. 222, 763-785.

86. Bjorkman & Mowbray (1998) J. Mol. Biol. 279, 651-664.

87. Wisz & Hellinga (2003) Proteins 51, 360-377.

88. Dwyer et al. (2003) Proc. Natl. Acad. Sci. USA 100, 11255-11260.

89. Mayo et al. (1990) J. Phys. Chem. 94, 8894-8909.

90. Ho et al. (1989) Gene 77, 51-59.

91. Bjorkman et al. (1994) J. Biol. Chem. 269, 30206-30211.

92. Vyas et al. (1988) Science 242, 1290-1295.

93. Jencks (1981) Proc. Natl. Acad. Sci. USA 78,4046-4050.

94. DeLano (2002) Curr. Opin. Struct. Biol. 12, 14-20.

95. Marvin & Hellinga (2001) Nat. Struct. Biol. 8, 795-798.

96. Millet et al. (2003) Proc. Natl. Acad. Sci. USA 100, 12700-12705.

97. Kuntz et al. (1999) Proc. Natl. Acad. Sci. USA 96, 9997-10002.

98. White (1994) Annu. Rev. Biophys. Biomol. Struct. 23, 407-439.

99. Wolfenden & Snider (2001) Acc. Chem. Res. 34, 938-945.

100. Benkovic & Hammes-Schiffer (2003) Science 301, 1196-1202.

101. Garcia-Viloca et al. (2004) Science 303, 186-195.

102. Hilvert (2000) Annu. Rev. Biochem. 69, 751-793.

103. Knowles (1991) Nature 350, 121-124.

104. Fraenkel (1986) Annu. Rev. Biochem. 55, 317-337.

105. Richard & Amyes (2001) Curr. Opin. Chem. Biol. 5, 626-633.

106. Richard (1985) Biochemistry 24, 949-953.

107. Cleland et al. (1998) J. Biol. Chem. 273, 25529-25532.

108. Harris et al. (1997) Biochemistry 36, 14661-14675.

109. Kursula & Wierenga (2003) J. Biol. Chem. 278, 9544-9551.

110. Jogl et al. (2003) Proc. Natl. Acad. Sci. USA 100, 50-55.

111. Lolis & Petsko (1990) Biochemistry 29, 6619-6625.

112. Ferguson et al. (1998) Arch. Microbiol. 170, 209-218.

113. Sampson & Knowles (1992) Biochemistry 31, 8488-8494.

114. Dwyer et al. (2003) Proc. Natl. Acad. Sci. USA 100, 11255-11260.

115. Hermes et al. (1989) Gene 84, 143-151.

116. Zaccolo et al. (1996) J. Mol. Biol. 255, 589-603.

117. Raines et al. (1986) Biochemistry 25, 7142-7154.

118. Kuhlman et al. (2003) Science 302, 1364-1368.

119. Schellman (1955) Compt. Rend. Trav. Lab. Carlsberg Ser. Chim. 29, 230-259.

120. Segel (1975) Enzyme Kinetics (Wiley, New York).

121. Hall & Knowles (1975) Biochemistry 14, 4348-4353.

122. Walsh (1995) Enzyme Reaction Mechanisms (Freeman, New York).

All documents cited herein are incorporated by reference in their entirety.

In stating a numerical range, it should be understood that all values within the range are also described (e.g., one to ten also includes every integer value between one and ten as well as all intermediate ranges such as two to ten, one to five, and three to eight). The term “about” may refer to the statistical uncertainty associated with a measurement or the variability in a numerical quantity which a person skilled in the art would understand does not affect operation of the invention or its patentability.

All modifications and substitutions that come within the meaning of the claims and the range of their legal equivalents are to be embraced within their scope. A claim using the transition “consisting” allows the inclusion of other elements to be within the scope of the claim; the invention is also described by such claims using the transitional phrase “consisting essentially of”(i.e., allowing the inclusion of other elements to be within the scope of the claim if they do not materially affect operation of the invention) and the transition “consisting” (i.e., allowing only the elements listed in the claim other than impurities or inconsequential activities which are ordinarily associated with the invention) instead of the “comprising” term. Any of these three transitions can be used to claim the invention.

It should be understood that an element described in this specification should not be construed as a limitation of the claimed invention unless it is explicitly recited in the claims. Thus, the granted claims are the basis for determining the scope of legal protection instead of a limitation from the specification which is read into the claims. In contradistinction, the prior art is explicitly excluded from the invention to the extent of specific embodiments that would anticipate the claimed invention or destroy novelty.

Moreover, no particular relationship between or among limitations of a claim is intended unless such relationship is explicitly recited in the claim (e.g., the arrangement of components in a product claim or order of steps in a method claim is not a limitation of the claim unless explicitly stated to be so). All possible combinations and permutations of individual elements disclosed herein are considered to be aspects of the invention. Similarly, generalizations of the invention's description are considered to be part of the invention.

From the foregoing, it would be apparent to a skilled person that the invention can be embodied in other specific forms without departing from its spirit or essential characteristics. The described embodiments should be considered only as illustrative, not restrictive.

TABLE 1
Complementary Surface Sequences of the Designated Receptors

| Scafold | Target | Design | Complementary Surface Seguence^{†} | | | | | | | |
|---|---|---|---|---|---|---|---|---|---|---|
| RBP | | | 9_{I} | 13_{I} | 15_{I} | 16_{I} | 64_{I} | 89_{I} | 90_{I} | 103_{H} |
| | | wt | S | N | F | F | N | D | R | S |
| | TNT | R1(I) | S | N | A | N | S | S | R | |
| | | R2(I) | S | I | A | N | N | A | D | |
| | | R3(I) | | S | F | L | | S | S | S |
| | L-lac | R1(A) | | V | A | R | | S | S | S |
| GPB | | | 10_{I} | 14_{I} | 16_{I} | 91_{I} | 92_{I} | 152_{II} | 154_{II} | 158_{II} |

| | | | | | | | | | |
|---|---|---|---|---|---|---|---|---|---|
| | wt | Y | D | F | N | K | H | D | R |
| L-lac | G1(A) | K | K | F | K | L | M | H | K |
| | G2(A) | K | M | K | K | L | K | K | M |
| D-lac | G1(A) | H | M | F | A | H | N | H | R |
| | G2(A) | Q | R | F | V | N | N | H | R |
| | G3(A) | K | M | S | V | S | N | H | R |

| | | | | | | | | |
|---|---|---|---|---|---|---|---|---|
| ABP | 10_{I} | 11_{I} | 14_{I} | 16_{I} | 17_{I} | 20_{I} | 64_{I} | 89_{I} |

| | | | | | | | | | |
|---|---|---|---|---|---|---|---|---|---|
| | wt | K | Q | E | W | F | E | C | D |
| TNT | A1(I) | S | R | T | A | A | K | A | A |
| | A2‡(I) | S | R | T | A | A | K | S | A |
| L-lac | A1(A) | K | | E | Y | | | A | L |
| | A2(A) | K | | L | Y | | | A | S |
| Stn | A1(I) | A | Q | E | A | S | Q | S | D |
| | A2‡(I) | A | Q | E | A | S | Q | S | D |

| HBP | | | 11_{I} | 14_{I} | 52_{I} | 70_{I} | 71_{I} | 72_{I} | 77_{I} | 117_{II} |
|---|---|---|---|---|---|---|---|---|---|---|
| | | wt | D | Y | L | S | L | S | R | L |
| | TNT | H1(I) | T | S | S | | F | K | R | A |
| | | H2(I) | N | A | N | | Q | K | R | L |
| | L-lac | H1(A) | | S | L | S | | K | R | K |
| | | h2(A) | | S | K | T | | K | R | H |
| QBP | | | 10_{I} | 13_{I} | 50_{I} | 70_{I} | 75_{I} | 115_{II} | 118_{II} | 156_{II} |
| | | wt | D | F | F | T | R | K | T | H |
| | L-lac | Q1(A) | D | L | L | S | R | K | T | H |
| | | Q2(A) | D | R | S | T | R | K | S | H |
| | | Q3(A) | D | L | K | S | D | K | T | H |

| Scafold | Target | Design | Complementary Surface Seguence^{†} | | | | | | | | | |
|---|---|---|---|---|---|---|---|---|---|---|---|---|
| RBP | | | 132_{H} | 137_{H} | 141_{II} | 164_{II} | 190_{II} | 214_{II} | 215_{II} | 235_{II} | | |
| | | wt | I | A | R | F | N | F | D | Q | | |
| | TNT | R1(I) | | | R | S | N | | A | S | | |
| | | R2(I) | K | | A | N | N | 214 | A | N | | |
| | | R3(I) | | S | S | I | F | | S | S | | |
| | L-lac | R1(A) | | K | M | K | I | | S | T | | |
| GPB | | | 183_{II} | 211_{II} | 236_{II} | 256_{II} | | | | | | |
| | | wt | W | N | D | N | | | | | | |
| | L-lac | G1(A) | K | N | A | D | | | | | | |
| | | G2(A) | K | N | A | S | | | | | | |
| | D-lac | G1(A) | Y | S | W | K | | | | | | |
| | | G2(A0 | Y | S | A | K | | | | | | |
| | | G3(A) | Y | S | A | Q | | | | | | |
| ABP | | | 90_{I} | 108_{II} | 145_{II} | 147_{II} | 151_{II} | 177_{II} | 204_{II} | 205_{II} | 232_{II} | 235_{II} |
| | | wt | D | M | L | T | R | N | M | N | N | D |
| | TNT | A1(I) | I | R | Q | S | D | L | A | | F | T |
| | | A2‡(I) | I | R | Q | S | D | L | A | | F | T |
| | L-lac | A1(A) | A | A | R | S | S | | T | N | K | |
| | | A2(A) | S | A | R | S | S | | T | K | H | |
| | Stn | A1(I) | D | E | L | T | R | E | M | N | N | |
| | | A2‡(I) | N | E | L | T | R | E | M | N | N | |
| HBP | | | 120_{II} | 121_{II} | 122_{II} | 143_{II} | 161_{II} | | | | | |
| | | wt | T | T | Q | Q | D | | | | | |
| | TNT | H1(I) | S | | | Q | S | | | | | |
| | | H2(I) | T | | Q | T | N | | | | | |
| | L-lac | H1(A) | S | S | T | | S | | | | | |
| | | h2(A) | S | A | T | | T | | | | | |
| QBP | | | 157_{II} | 185_{II} | | | | | | | | |
| | | wt | D | Y | | | | | | | | |
| | L-lac | Q1(A) | H | F | | | | | | | | |
| | | Q2(A) | R | F | | | | | | | | |
| | | Q3(A) | R | F | | | | | | | | |
| *The nomenclature for the designed receptors gives the target ligand and a single letter abbreviation of the scaffold protein. | | | | | | | | | | | | |
| ^{†}Subscripts indicate the location of a residue position: I, N-terminal domain; II, C-terminal domain; H, hinge (FIG. 2). Bold letters indicate mutations from wild-type (calculations may predict retention of wild-type residues). Underlined letters represent side-chains making hydrogen bonds with the ligand (cognate ligand in the case of wt). The design calculations in a given receptor were not always carried out with identical PCS residues: automated identification of | | | | | | | | | | | | |
| # the PCS is in dicated by “A”; “I” indicates identification by inspection. Blanks therefore indicate residue positions not included in a calculation (wild-type sequence and conformation). | | | | | | | | | | | | |

‡TNT.A2 is a point mutant of TNT.A1; Stn.A2 is a point mutant of Stn.A1. Both mutations were designed by inspection.

TABLE 2
Affinities of the Designed Receptors for Target Ligands and
Analogs

| | K₁ (μM)* | | | | |
|---|---|---|---|---|---|
| Target | Receptor | TNT | TNB | 2.4-DNT | 2.6-DNT |
| TNT | RBP | | | | |
| | R1 | 0.34 | 1.0 | 5.0 | 5.4 |
| | R2 | 1.6 | 3.8 | 5.3 | 4.9 |
| | R3 | 0.002 | 0.1 | 8.4 | 15 |
| | ABP | | | | |
| | A1 | 1400 | 600 | >10000 | >10000 |
| | A2 | 400 | 500 | 2000 | 4000 |
| | HBP | | | | |
| | H1 | 220 | 1000 | >10000 | >10000 |
| | H2 | 200 | 800 | >10000 | >10000 |
| | | L-lac | | D-lac | Pyr |
| L-lactate | GBP | | | | |
| | G1 | 2.8 | | 205 | 255 |
| | G2 | 2.1 | | 55 | 115 |
| | HBP | | | | |
| | H1 | 1.8 | | 40 | 50 |
| | H2 | 12.2 | | 30 | 48 |
| | QBP | | | | |
| | Q1 | 9500 | | >100000 | >100000 |
| | Q2 | 300 | | >100000 | >100000 |
| | Q3 | 25000 | | >100000 | >100000 |
| | ABP | | | | |
| | A1 | 160 | | >100000 | >100000 |
| | A2 | 20000 | | >100000 | >100000 |
| | RBP | | | | |
| | R1 | 7.4 | | 40 | 40 |
| | | D-lac | | L-lac | Pyr |
| D-lactate | GBP | | | | |
| | G1 | 0.8 | | 10 | 55 |
| | G2 | 1.5 | | 24 | 65 |
| | G3 | 2 | | 17 | 22 |
| | | Stn | | Trp | Trm |
| Serotonin | ABP | | | | |
| | A1 | 50 | | 660 | 900 |
| | A2 | 4.7 | | 65 | 90 |

*The limit of detection for the nitro compounds corresponds to affinities of approximately 10 mM, and for lactate analogues to 100 mM. Error of K_{d} measurement is approximately 10%.
Abbreviations:
TNB, trinitrobenzene;
DNT, dinitrobenzene;
Pyr, pyruvate;
Stn, serotonin;
Trp, L-tryptophan;
Trm, tryptamine

TABLE 3
Sequence and Binding Properties of the Designed Receptors

Complementary Surface Sequences*

| Design | 10_{I} | 14_{I} | 16_{I} | 91_{I} | 92_{I} | 152_{II} | 154_{II} | 158_{II} | 183_{II} | 211_{II} | 236_{II} | 256_{II} |
|---|---|---|---|---|---|---|---|---|---|---|---|---|
| wtGBP | Y | D | F | N | K | H | D | R | W | N | D | N |
| PG4 | Q | K | F | A | S | N | N | I | H | S | A | S |
| PG4_256F | Q | K | F | A | S | N | N | I | H | S | A | F |
| PG5^{∥} | Q | K | F | S | S | N | S | S | Y | S | A | S |
| PG6^{∥} | K | H | H | A | S | N | S | I | F | S | A | S |
| PG7^{∥} | Q | S | L | V | S | N | S | Q | F | S | A | H |
| PG8^{∥} | Q | K | F | A | S | N | S | S | Y | S | A | S |
| PG9 | Q | K | F | A | S | N | H | I | H | S | A | S |
| PG10 | K | M | F | A | S | N | Y | I | K | S | A | S |
| PG11** | K | S | H | V | S | N | S | Q | Y | S | A | S |
| PG12 | Q | K | F | S | S | N | S | I | H | N | A | S |
| PG12_256F | Q | K | F | S | S | N | S | I | H | N | A | F |
| PG14^{†}^{†} | M | R | F | S | S | N | H | K | F | S | A | H |
| PG17 | Q | K | F | S | S | N | S | I | Y | N | A | S |
| PG18 | Q | K | F | A | S | N | S | I | Y | S | A | S |
| wtRBP | N | F | F | D | R | A | R | F | N | D | Q | |
| PR8 | S | A | S | S | D | A | M | K | K | A | S | |
| PR9^{∥} | N | F | L | N | D | S | M | H | S | A | S | |
| PR11^{∥} | N | F | L | N | D | S | M | S | S | A | S | |
| PR8_235A | S | A | S | S | D | A | M | K | K | A | A | |
| PR8_235L | S | A | S | S | D | A | M | K | K | A | L | |
| PR8_235F | S | A | S | S | D | A | M | K | K | A | F | |

| | | | | |
|---|---|---|---|---|
| PMPA^{†‡} | PA^{†} | MP^{†} | Fluoro- | PMPA |

| Design | K_{d}(μM) | K_{d}(mM) | K_{d}(mM) | phores^{‡‡} | ΔI_{std}^{¶} |
|---|---|---|---|---|---|
| wtGBP | nb | | | IAF | 0 |
| PG4 | 0.7 | 0.04 | 14 | IAF | 0.041 (−) |
| PG4_256F | 0.4 | nb | 12 | IAF | 0.015 (−) |
| PG5^{∥} | nb | | | | 0 |
| PG6^{∥} | nb | | | | 0 |
| PG7^{∥} | nb | | | | 0 |
| PG8^{∥} | nb | | | | 0 |
| PG9 | 3.4 | 0.3 | 10 | IAF | 0.11 (+) |
| PG10 | 0.44 | 1.5 | 12 | IAF | 0.22 (−) |
| PG11** | | | | | |
| PG12 | 0.25 | 0.8 | 9.5 | IAF | 0.15 (+) |
| PG12_256F | 0.11 | 1.3 | 11 | IAF | 0.23 (−) |
| PG14^{†}^{†} | | | | | |
| PG17 | 5 | nb | 140 | NBDE | 0.059 (−) |
| PG18 | 10 | 8.7 | nb | NBDE | 0.014 (−) |
| wtRBP | nb | | | | 0 |
| PR8 | 0.068 | | | JPW4042 | 0.082 (+) |
| PR9^{∥} | nb | | | | 0 |
| PR11^{∥} | nb | | | | 0 |
| PR8_235A | 0.069 | | | JPW4039 | 0.08 (−) |
| PR8_235L | 0.064 | | | JPW4039 | 0.07 (−) |
| PR8_23DF | 0.045 | | | JPW4039 | 0.063 (−) |

*Amino acids are given in one-letter abbreviation. Subscripts indicate the location of the residue: I, N-terminal domain; II C-terminal domain. Bold, mutation from wild-type; underlined, hydrogen bond to PMPA
^{†}nb: no binding as determined by a fluorescence change at maximum ligand concentrations (PMPA, 10 mM; PA, 100 mM; MP, 1000 mM). Blank entry: not determined
^{‡}Affinities were determined using a racemic mixture of PMPA. The receptors were designed for the R-isomer
^{§}Fluorophores (IAF, NBDE and Acrylodan) used to test the design for binding to PMPA; fluorophore used in determining affinity is presented in the table
^{¶}Fluorescence emission intensity change in the presence of saturating ligand (ΔI_{std} as defined in de Lorimier et al. (7))
^{∥}No ligand-mediated change in fluorescence upon addition of PMPA (10 mM)
**Protein precipitated
^{††}No expression
^{‡‡}Fluorophores (JPW4039, JPW4042 and JPW4045) used to test the design for binding to PMPA; fluorophore used in determining affinity is presented in the table

TABLE 4
Alanine Point Mutations in PG10 and PG12

| | PMPA | | PA | | MP | | |
|---|---|---|---|---|---|---|---|
| | | ΔΔG^{†} | | ΔΔG^{†} | | ΔΔG^{†} | |
| Design* | K_{d} (μM) | (kcal/mol) | K_{d} (mM)^{‡} | (kcal/mol) | K_{d} (mM)^{‡} | (kcal/mol) | Interaction^{§} |
| PG10 | 0.45 | | 1.3 | | 12 | | |
| PG10_S92A | 19 | 2.2 | 0.29 | −0.87 | nb | nb | latch |
| PG10_N152A | 56 | 2.9 | 0.92 | −0.19 | 12 | 0 | latch |
| PG10_Y154A | 23 | 2.4 | 8.7 | 1.1 | 9.1 | −0.17 | van der Waals contact to pinacolyl |
| | | | | | | | group, and hydrogen bond to T110 |
| PG10_K183A | 12 | 2.0 | 0.32 | −0.81 | 17 | 0.21 | hydrogen bond to O3 |
| PG10_S211A | 16 | 2.1 | 1.8 | 0.21 | 11 | 0.06 | hydrogen bond to O1 |
| PG10_S256A | 0.6 | 0.02 | 1.6 | 0.12 | 11 | −0.08 | hydrogen bond to O2, and to Q162 |
| PG12 | 0.3 | | 0.8 | | 9.5 | | |
| PG12_S92A | 2 | 1 | 1 | 0.2 | 11 | 0.1 | latch |
| PG12_N152A | 7 | 2 | nb | | 8.5 | 0.06 | hydrogen bond to O1, and latch |
| PG12_S154A | 90 | 3 | nb | | 9.8 | 0.02 | hydrogen bond to O2 |
| PG12_H183A | 30 | 3 | nb | | 9.1 | −0.02 | hydrogen bond to O1 |
| PG12_N211A | 0.9 | 0.8 | 2 | 0.5 | 9.0 | −0.03 | hydrogen bond to M214 carbonyl, |
| | | | | | | | van der Waals contact to pinacolyl |
| PG12_S256A | 200 | 4 | nb | | 17 | 0.4 | group hydrogen bond to Q261 |

*Labeled with IAF
^{†}ΔΔG = −RT ln K_{d}^{Design}/K_{d}^{Mutant}
^{‡}No binding (nb) at maximal ligand concentrations (PA, 100 mM; MP, 1000 mM)
^{§}See FIG. 9 for oxygen numbering scheme

TABLE 5
Sequences of the Designed Receptors*

| Protein^{†} | 9_{I} | 10_{I} | 13_{I} | 15_{I} | 16_{I} | 41_{I} | 64_{I} | 89_{I} | 90_{I} | 132_{II} | 135_{II} |
|---|---|---|---|---|---|---|---|---|---|---|---|
| wt RBP | S | T | N | F | F | N | N | D | R | I | T |
| D.1 | | | n | Y | f | | | S | N | | |
| .2 | | | n | H | f | | | G | K | | |
| .3 | | | K | f | L | | | G | S | | |
| .4 | | | K | f | Q | | | S | S | | |
| NTim1.0 | | | S | E | H | | | A | H | K | |
| 1b | | | S | E | f | | | A | H | K | |
| 1c | | | S | E | H | | | A | H | K | |
| 1d | | | S | E | H | | | A | H | K | |
| 1e | | | S | E | H | | | A | H | K | |
| 2a | S | E | H | f | K | S | S | d | H | K | |
| 2b | S | E | H | f | K | S | S | d | H | K | |
| 2c | S | E | S | Q | S | S | K | A | H | K | |
| 2d | G | E | H | f | K | K | S | d | H | K | |
| 2e | G | E | H | f | K | S | S | d | H | K | |
| 3a | S | T | A | | H | | T | W | H | V | E |
| 3b | S | T | A | | H | | T | W | N | V | E |
| 3c | S | T | A | | H | | D | W | H | V | E |
| 3d | S | T | A | | H | | M | W | H | V | E |
| NTim1.1§ | | H | S | E | H | | T | A | H | K | |
| NTim1.2§ | | N | S | E | H | | H | D | H | K | |

| Protein^{†} | 137_{II} | 138_{II} | 141_{II} | 164_{II} | 189_{II} | 190_{II} | 192_{I} | 214_{II} | 215_{II} | 235_{II} | Activity‡ |
|---|---|---|---|---|---|---|---|---|---|---|---|
| wt RBP | A | A | R | F | Q | N | E | F | D | Q | − |
| D.1 | G | | H | K | | S | | | G | q | − |
| .2 | S | | K | K | | S | | | S | M | − |
| .3 | S | | M | K | | T | | | A | V | − |
| .4 | S | | K | L | | S | | | A | q | − |
| NTim1.0 | S | | K | G | | G | N | f | S | q | +++ |
| 1b | S | | K | G | | G | E | f | A | T | + |
| 1c | S | | K | G | | G | N | f | T | q | + |
| 1d | S | | K | G | | G | E | f | T | S | + |
| 1e | S | | K | G | | G | E | f | S | q | + |
| 2a | K | | K | G | | G | | | A | | − |
| 2b | K | | K | G | | G | | | L | | + |
| 2c | K | | K | G | | G | | | A | | − |
| 2d | K | | K | G | | G | | | A | | − |
| 2e | K | | K | G | | G | | | A | | − |
| 3a | G | S | K | G | L | K | | | | | + |
| 3b | G | S | K | G | L | K | | | | | − |
| 3c | G | S | K | G | L | K | | | | | − |
| 3d | G | S | K | G | L | K | | | | | − |
| NTim1.1§ | S | a | K | G | | G | N | f | S | q | +++ |
| NTim1.2§ | S | a | K | G | | G | E | f | N | S | ++++ |

*Subscripts indicate the location of a residue position (FIG. 15C): I, N-terminal domain; II, C-terminal domain; H, hinge; blank entry: position not included in the calculation; lower case: wild-type residue retained in the calculation; catalytic residues in bold; underlined letters side-chains making hydrogen bonds with the substrate (ribose in the case of wild-type RBP) are underlined;
^{†}DHAP- and GAP-binding receptors, D.1-4; first-round designed enzymes (NovoTims) are divided into three families, Ntim1(b-e), 2(a-e), 3(a-d). NovoTim1.0 is the most active first-round design. NovoTim 1.1 and NovoTim 1.2 are improvements based on NovoTim, with a secondary layer of residues (included in calculation, but not tabulated: 1.1, N105Y, R166, T217K, L265L; 1.2, V8I, N105Y, R166R, L265L).
‡Semi-quantitative assessment of activity based on end-point analysis of the forward (DHAP to GAP) reaction: −, background (uncatalyzed); +, >5-fold increase after 6 hr; +++, >10-fold after 5 min; ++++, >20-fold increase after 5 min

TABLE 6
Kinetic Parameters for Forward and Reverse Isomerization Reactions

| | Protein | | | | | | | | |
|---|---|---|---|---|---|---|---|---|---|
| | | | | | DHAP [symbol] GAP | | | | |
| | DHAP → GAP | | | | | | | k_{cat}/ | |
| NovoTim | k_{cat} (s⁻¹) | K_{M} (μM) | k_{cat}/K_{M} (M⁻¹s⁻¹) | k_{cat}/k_{uncat} | k_{cat}(s⁻¹) | K_{M} (μM) | k_{cat}/K_{M} (M⁻¹s⁻¹) | k_{uncat}^{§} | ^{app}K_{eq}^{†} |
| 1.0 | 0.05 | 330 | 1.5 × 10² | 2.4 × 10⁵ | nd* | nd | nd | nd | nd |
| 1.2 | 0.1 | 180 | 5.6 × 10² | 5.0 × 10⁵ | 0.8 | 92 | 8.6 × 10³ | 1.8 × 10⁵ | 15 |
| 1.2.1 | 0.18 | 140 | 1.4 × 10³ | 9.0 × 10⁵ | 1.5 | 85 | 1.7 × 10⁴ | 3.4 × 10⁵ | 12 |
| 1.2.2 | 0.14 | 165 | 8.2 × 10² | 7.0 × 10⁵ | 1.2 | 89 | 1.4 × 10⁴ | 2.7 × 10⁵ | 17 |
| 1.2.3 | 0.17 | 100 | 1.8 × 10³ | 8.5 × 10⁵ | 1.2 | 103 | 1.2 × 10⁴ | 2.7 × 10⁵ | 7 |
| 1.2.4 | 0.11 | 105 | 1.0 × 10³ | 5.5 × 10⁵ | 1.1 | 51 | 2.1 × 10⁴ | 2.5 × 10⁵ | 21 |
| wtTIM^{‡} | 487 | 1600 | 3.0 × 10⁵ | >1 × 10⁹ | 4.3 × 10³ | 390 | 1.0 × 10⁷ | 1.0 × 10⁹ | 33 |

*nd, not determined.
^{†}^{app}K_{eq} is the apparent equilibrium constant (Haldane constant (18)) calculated from the ratio k_{cat}/KM values of the forward and reverse reactions
^{‡}wild type parameters from (103)
^{§}uncatalyzed reaction rate from (121)

1. A process for protein design in accordance with spatial and energy relationships between a proteinaceous receptor and a ligand, the process comprising:
(a) generating a collection of ligand poses to provide a Docking Zone which represents potential conformation and degrees of freedom of the ligand relative to the receptor,
(b) generating a collection of side-chain conformations on the receptor's backbone to provide an Evolving Zone which represents potential receptor mutants,
(c) constructing a cost function from atomic interaction(s) between the ligand poses of the Docking Zone and the side chains of the Evolving Zone and between side chains of the Evolving Zone, and
(d) selecting one or more combinations of single ligand pose and cognate receptor mutant which correspond to optimal or near-optimal values of the cost function to generate a collection of potential receptor mutants with ligand-binding sites, wherein the protein designed by the process is a potential receptor mutant.

2. The process according to claim 1 further comprising (e) rank-ordering ligand-binding sites of potential receptor mutants by a fitness metric prior to confirming whether or not one or more receptor mutants bind to the ligand or an analog thereof.

3. The process according to claim 2, wherein the fitness metric comprises one or more descriptors selected from the group consisting of a semi-empirical or universal force field, solvent-accessible area, cavity volume, ligand affinity, and ligand reactivity.

4. The process according to claim 1, wherein only a subset of all possible combinations between ligand poses of the Docking Zone and side chains of the Evolving Zone in at least (d) are further evaluated.

5. The process according to claim 4 further comprising evaluation of the hydrogen bond inventory for at least one ligand pose of the Docking Zone.

6. The process according to claim 4 further comprising evaluation of a binding surface inventory for atomic interaction(s) between at least one ligand pose of the Docking Zone and at least one side chain of the Evolving Zone.

7. The process according to claim 1, wherein all possible combinations between ligand poses of the Docking Zone and side chains of the Evolving Zone in at least (d) are further evaluated.

8. The process according to claim 1 further comprising introducing additional mutations in the designed protein and selecting a re-designed protein for at least one of increased stability, increased affinity, and increased catalytic activity.

9. A process for manufacturing a protein, wherein the process comprises expressing and isolating the one or more receptors predicted by claim 1 to bind the ligand.

10. A computer system, wherein the process of claim 1 is implemented as instructions for manipulating data by the computer system.

11. A tangible medium, wherein the process of claim 1 is stored thereon as software.

12. A protein designed by the process according to claim 1.

13. A protein produced by the process according to claim 9.

14. The protein of claim 13, wherein the protein is comprised of an amino acid sequence selected from the group consisting of mutant receptors listed in Tables 1, 3 and 5.

15. The protein of claim 12 or, wherein the ligand confers allosteric regulation on protein activity.

16. A catalyst comprised of the protein of claim 12 or.

17. An affinity or chiral purification reagent comprised of the protein of claim 12.

18. A biosensor comprised of the protein of claim 12.

19. A nucleic acid which encodes the protein of claim 12.

20. An expression vector comprised of the nucleic acid of claim 19.

21. An engineered cell, tissue, or non-human organism which expresses the protein of claim 12.

22. The engineered cell, tissue, or non-human organism of claim 21, wherein the protein is in at least one of a signal transduction pathway, a genetic circuit, or a metabolic pathway.

23. An engineered cell, tissue, or non-human organism which is comprised of the nucleic acid of claim 19.

24. The engineered cell, tissue, or non-human organism of claim 23, wherein the protein is in at least one of a signal transduction pathway, a genetic circuit, or a metabolic pathway.

25. An engineered cell, tissue, or non-human organism which is comprised of the expression vector of claim 20.

26. The engineered cell, tissue, or non-human organism of claim 25, wherein the protein is in at least one of a signal transduction pathway, a genetic circuit, or a metabolic pathway.


SEQUENCE LISTING (5 sequences)

SEQ ID NO: 1 - protein, 309 aa, Escherichia coli [sequence omitted]

SEQ ID NO: 2 - protein, 271 aa, Escherichia coli
KDTIALVVSTLNNPFFVSLKDGAQKEADKLGYNLVVLDSQNNPAKELANVQDLTVRGTKILLINPTDSDAVGNAVKMANQANIPVITLDRQATKGEVVSHIASDNVLGGKIAGDYIAKKAGEGAKVIELQGIAGTSAARERGEGFQQAVAAHKFNVLASQPADFDRIKGLNVMQNLLTAHPDVQAVFAQNDEMALGALRALQTAGKSDVMVVGFDGTPDGEKAVNDGKLAATIAQLPDQIGAKGVETADKVLKGEKVQAKYPVDLKLVVKQ

SEQ ID NO: 3 - protein, 306 aa, Escherichia coli
ENLKLGFLVKQPEEPWFQTEWKFADKAGKDLGFEVIKIAVPDGEKTLNAIDSLAASGAKGFVICTPDPKLGSAIVAKARGYDMKVIAVDDQFVNAKGKPMDTVPLVMMAATKIGERQGQELYKEMQKRGWDVKESAVMAITANELDTARRRTTGSMDALKAAGFPEKQIYQVPTKSNDIPGAFDAANSMLVQHPEVKHWLIVGMNDSTVLGGVRATEGQGFKAADIIGIGINGVDAVSELSKAQATGFYGSLLPSPDVHGYKSSEMLYNWVAKDVEPPKFTEVTDVVLITRDNFKEELEKKGLGGK

SEQ ID NO: 4 - protein, 226 aa, Escherichia coli
ADKKLVVATDTAFVPFEFKQGDKYVGFDVDLWAAIAKELKLDYELKPMDFSGIIPALQTKNVDLALAGITITDERKKAIDFSDGYYKSGLLVMVKANNNDVKSVKDLDGKVVAVKSGTGSVDYAKANIKTKDLRQFPNIDNAYMELGTNRADAVLHDTPNILYFIKTAGNGQFKAVGDSLEAQQYGIAFPKGSDELRDKVNGALKTLRENGTYNEIYKKWFGTEPK

SEQ ID NO: 5 - protein, 238 aa, Escherichia coli
AIPQNIRIGTDPTYAPFESKNSQGELVGFDIDLAKELCKRINTQCTFVENPLDALIPSLKAKKIDAIMSSLSITEKRQQEIAFTDKLYAADSRLVVAKNSDIQPTVESLKGKRVGVLQGTTQETFGNEHWAPKGIEIVSYQGQDNIYSDLTAGRIDAAFQDEVAASEGFLKQPVGKDYKFGGPSVKDEKLFGVGTGMGLRKEDNELREALNKAFAEMRADGTYEKLAKKYFDFDVYGG